(19) United States
(12) Patent Application Publication
(10) Pub. No.: US 2024/0173408 A1
(43) Pub. Date: May 30, 2024

(54) PROGNOSTIC BIOMARKERS FOR CANCER RELAPSE VACCINATION AND THE USE THEREOF

(71) Applicant: MENDUS B.V., Leiden (NL)

(72) Inventors: Jeroen Rovers, Leiden (NL); Satwinder Kaur Singh, Leiden (NL); Hester van Zeeburg, Leiden (NL)

(21) Appl. No.: 18/499,357

(22) Filed: Nov. 1, 2023

Related U.S. Application Data
(60) Provisional application No. 63/425,366, filed on Nov. 15, 2022.
(60) Provisional application No. 63/382,080, filed on Nov. 2, 2022.

(51) Int. Cl. A61K 39/00; A61P 35/02; G01N 33/574

(52) U.S. Cl. CPC A61K 39/4622; A61K 39/4615; A61K 39/464429; A61K 39/46445; A61K 39/464453; A61K 39/46447; A61K 39/464489; A61P 35/02; G01N 33/57426; G01N 33/57484; A61K 2239/26; A61K 2239/31; A61K 2239/48

(57) ABSTRACT

Disclosed provides a method of treating measure residue disease (MRD) in a subject with cancer using an allogeneic leukemia-derived cell as a vaccine based on the information provided by prognostic biomarkers comprising dendritic cells including cDC1 cDC2, and/or pDC; CD8+ T cells including CD8+CD45RA+ cells, CD8+CD45RA− CCR7+CM T cells, and/or CD8 RO+ T cells; B cells; NK cells including CD56++NK cells and/or CD56+NK cells; CD4 CD161+ T cells; CD14+CD16− non-inflammatory monocytes, or any combination thereof.

### RELATED APPLICATIONS

This application claims the benefit of U.S. Provisional Application No. 63/382,080, filed on Nov. 2, 2022, and 63/425,366, filed on Nov. 15, 2022, each of which is incorporated herein by reference in its entirety.

### FIELD OF THE INVENTION

The present disclosure relates to prognostic biomarkers for cancer relapse vaccination and methods of treating cancer with an allogeneic leukemia-derived cell as a vaccine based on the information provided by biomarkers.

### BACKGROUND

While the advent of targeted therapies has improved survival in certain cancer subtypes, relapse after initial therapy is a major problem. Initial cancer treatments such as radiation therapy, chemotherapy, surgery, in most cases, modulate the immune system, either or not in combination with an aging immune system. Because the level of immune competence frequently is usually suppressed, damaged, or reduced due to the cancer itself or initial cancer treatments, cancer vaccination, or cancer immunotherapy, to direct the immune system to eradicate cancer cells and prevent relapse of cancer, both solid and hematological malignancies, is challenging. Leukemias present the additional challenge of severely disrupted hematopoiesis due to both cytogenic defects in hematopoietic progenitors and an abnormal hematopoietic stem cell niche in the bone marrow, which accentuate systemic immunosuppression and DC malfunction. (LJ O'Brien, et al. Cancers (Basel). 2019 Jun:11(6): 875).

Measurable residual disease, also called minimal residual disease, (MRD) is a condition where a very small number of cancer cells remain in the body during or after an initial treatment and may not be detected with conventional morphologic methods, including standard histology, pathology, or diagnostic imaging. MRD usually can be found only by highly sensitive laboratory methods that are able to find one cancer cell in about 10,000 to about one million cells in blood samples.

Vaccination strategies for cancer treatment rely on successful processing and presentation of introduced antigens to initiate an adaptive immune response able to kill residual cancer cells, where processing of antigens is optimally done by professional antigen presenting cells, such as dendritic cells, macrophages, or monocytes. Dendritic cells (DC) are uniquely able to induce naïve T cell activation and effector differentiation. They are, likewise, involved in the induction and maintenance of immune tolerance in homeostatic conditions. Their phenotypic and functional heterogeneity points to their great plasticity and ability to modulate, according to their microenvironment, the acquired immune response and, at the same time, makes their precise classification complex and frequently subject to reviews and improvement.

Priming the immune system to eradicate or control residual disease using dendritic cells as vaccine could be an effective strategy as maintenance therapy in MRD positive patients to prevent relapse. Although cancer vaccination involving the use of dendritic cells (DC) has provided potential to induce tumor-specific T cells to provide anti-tumor immunity, currently, there is still a lack of methods to predict clinic response to the cancer vaccination. As such, there is a need to develop clinical biomarkers to predict the clinical response to the cancer vaccination in order to improve treatment and design the treatment regimen to achieve better results in preventing or delaying relapse of cancer.

### SUMMARY

The present disclosure is based, at least in part, on the discovery that the levels of certain immune cells, such as certain subsets of dendritic cells, correlate with the clinical response induced by an allogeneic leukemia-derived cell based vaccine (e.g., DCP-001) when DCP-001 is administered to an AML patient with MRD. DCP-001 is a vaccine derived from the DCOne leukemic cell line, DCOne cells of which can adopt a highly immunogenic mature dendric cell (mDC) phenotype. DCOne cells express multiple common tumor-associated antigens. DCOne mDC combines the DCOne tumor-associated antigen repertoire with a mDC costimulatory profile and this forms the basis for DCP-001, which is a frozen, cell-based, irradiated product, administered as an intradermal vaccine.

In one aspect, provided here is a method of treating cancer in a subject in need thereof, comprising administering to a subject having measurable residual disease (MRD) and an elevated level of a biomarker in peripheral blood of the subject relative to a reference level of the biomarker, a composition comprising an allogeneic leukemia-derived cell, wherein the biomarker may be chosen from dendritic cells, CD8+CD45RA+ cells, CD4 CD161+ T cells, CD14+CD16− non-inflammatory monocytes, or a combination thereof.

In various exemplary embodiments, the dendritic cells may be conventional dendritic cells DC1 (cDC1) and/or conventional DC2 (cDC2). In some embodiments, the dendritic cells may be CD141+/CLEC9A+ cDC1, cDC2, CD163+ cDC2, HLA-DR+/CD123+ pDC cells, or combination thereof. In some embodiments, the NK cells may be CD56++NK cells and/or CD56+NK cells.

In some embodiments, the cancer is a liquid cancer. In some further embodiments, the liquid cancer is selected from the group consisting of leukemia, lymphoma, and myeloma. In certain embodiments, the liquid cancer is acute myeloid leukemia (AML).

In various exemplary embodiments the allogeneic leukemia-derived cell comprises the tumor-associated antigens WT-1, MUC-1, PRAME, RHAMM, P53, and Survivin.

In various exemplary embodiments, the allogeneic leukemia-derived cell comprises a dendritic cell phenotype. In some embodiments, the allogeneic leukemia-derived cell comprises a mature dendritic cell phenotype. In certain exemplary embodiments, the allogeneic leukemia-derived cell comprises a genetic aberration between chromosome 11p15.5 to 11p12. In certain exemplary embodiments, the genetic aberration encompasses about 16 Mb of genomic regions.

In various exemplary embodiments, the allogeneic leukemia-derived cell is CD34-positive, CD1a-positive, and CD83-positive. In certain exemplary embodiments, the allogeneic leukemia-derived cell expresses a cell surface marker selected from the group consisting of DC-SIGN, Langerin, CD80, CD86, CD70, CD40, and any combination thereof. In certain embodiments, the allogeneic leukemia-derived cell is CD34-positive, CD1a-positive, CD83-positive, CD80-positive, CD86-positive, and CD40-positive. In some other embodiments, the allogeneic leukemia-derived cell may be CD14-negative. In certain exemplary embodiments, the allogeneic leukemia-derived cell is derived from the DCOne cell line.

In various exemplary embodiments, the subject receives one or more biweekly doses of about 25e6 or about 50e6 allogeneic leukemia-derived dendritic cells. In certain exemplary embodiments, the subject receives one or more booster doses, each dose comprising about 10e6 allogeneic leukemia-derived dendritic cells. In certain exemplary embodiments, each booster dose is administered to the subject about 28 days after a previous dose.

In various exemplary embodiments, the subject achieves MRD conversion (to negativity) or disappearance, or substantial reduction after receiving treatment with the composition comprising the allogeneic leukemia-derived dendritic cells.

In various exemplary embodiments, the composition is administered to the subject by intradermal injection.

In various exemplary embodiments, the subject had previously been treated by other therapy such as radiation therapy, chemotherapy, etc. In certain exemplary embodiments, the subject is in complete remission (CR).

In various exemplary embodiments, the subject has an elevated baseline level of HLA-DR+CD45RA+ dendritic cells relative to a reference level of the biomarker.

In another aspect, provided here is a method of treating cancer in a subject in in need thereof, comprising: administering to a subject having measurable residual disease (MRD) one or more initial doses of an allogeneic leukemia-derived dendritic cell vaccine; and administering to the subject one or more booster doses of the allogeneic leukemia-derived dendritic cell vaccine, if an elevated level of a biomarker is achieved in the subject subsequent to the one or more initial doses of the allogeneic leukemia-derived dendritic cell vaccine relative to the biomarker level in the subject prior to the one or more initial doses, wherein the biomarker comprises is chosen from dendritic cells, CD8+CD45RA+ cells, B cells, NK cells, CD4 CD161+ T cells, CD14+CD16− non-inflammatory monocytes, or a combination thereof.

In certain exemplary embodiments, the dendritic cells are chosen from cDC1 and/or cDC2. In certain exemplary embodiments, the dendritic cells comprise CD141+/CLEC9A⁺ cDC1 dendritic cells and/or CD163⁺ cDC2 dendritic cells. In some embodiments, the NK cells may be CD56++NK cells and/or CD56+NK cells.

In some embodiments, the cancer is a liquid cancer. In some further embodiments, the liquid cancer is selected from the group consisting of leukemia, lymphoma, and myeloma. In certain embodiments, the liquid cancer is acute myeloid leukemia (AML).

In various exemplary embodiments the allogeneic leukemia-derived cell comprises WT-1, MUC-1, PRAME, P53, RHAMM, and Survivin. In various exemplary embodiments, the allogeneic leukemia-derived cell comprises a dendritic cell phenotype. In some embodiments, the allogeneic leukemia-derived cell comprises a mature dendritic cell phenotype. In certain exemplary embodiments, the allogeneic leukemia-derived cell comprises a genetic aberration between chromosome 11p15.5 to 11p12. In certain exemplary embodiments, the genetic aberration encompasses about 16 Mb of genomic regions.

In various exemplary embodiments, the allogeneic leukemia-derived cell is CD34-positive, CD1a-positive, and CD83-positive. In certain exemplary embodiments, the allogeneic leukemia-derived cell expresses a cell surface marker selected from the group consisting of DC-SIGN, Langerin, CD80, CD86, CD70, CD40, and any combination thereof. In certain embodiments, the allogeneic leukemia-derived cell is CD34-positive, CD1a-positive, CD83-positive, CD80-positive, CD86-positive, and CD40-positive. In some other embodiments, the allogeneic leukemia-derived cell may be CD14-negative. In certain exemplary embodiments, the allogeneic leukemia-derived cell is derived from the DCOne cell line.

In various exemplary embodiments, the subject receives one or more biweekly doses of about 25e6 or about 50e6 allogeneic leukemia-derived dendritic cells. In certain exemplary embodiments, the subject receives one or more booster doses, each dose comprising about 10e6 allogeneic leukemia-derived dendritic cells. In certain exemplary embodiments, each booster dose is administered to the subject about 28 days after a previous dose.

In various exemplary embodiments, the subject achieves MRD conversion or disappearance after receiving treatment with the composition comprising the allogeneic leukemia-derived dendritic cells.

In various exemplary embodiments, the composition is administered to the subject by intradermal injection.

In various exemplary embodiments, the subject had previously been treated by other therapy such as radiation therapy, chemotherapy, etc. In certain exemplary embodiments, the subject is in complete remission (CR).

In various exemplary embodiments, the subject has an elevated baseline level of HLA-DR+CD45RA+ dendritic cells relative to a reference level of the biomarker.

In another aspect, provided here is a method of identifying a subject with cancer in remission having measurable residual disease (MRD) who is likely to be responsive to a treatment with an immunogenic composition, predicting a risk of developing cancer relapse or recurrence, and/or screening a candidate to receive the treatment with the immunogenetic composition, the method comprising: assessing a baseline level of a biomarker in a peripheral blood of the subject before the subject is treated with the immunogenetic composition in comparison with a predetermined reference level of the biomarker, where the predetermined reference level corresponds to a level similar to the baseline level in a subject who has relapse after receiving the immunogenetic composition; and identifying the subject as being likely to have a reduced MRD level or disappearance of the MRD in response to the treatment with the immunogenetic composition if the baseline level of the biomarker in the peripheral blood of the subject is greater or less than the predetermined reference level of the biomarker; wherein the immunogenetic composition comprises allogeneic leukemia-derived dendritic cells; and wherein the biomarker comprises at least one subset of circulating dendritic cells.

In various exemplary embodiments, the baseline level of the biomarker corresponds to the number of the at least one subset of dendritic cells in the peripheral blood of the subject and is assessed by measuring the number of the at least one subset of dendritic cells in a blood sample obtained from the subject before the subject is treated with the immunogenetic composition.

In certain exemplary embodiments, the predetermined level of the biomarker corresponds to a mean number of the at least one subset of dendritic cells in the peripheral blood of one or more separate subjects with cancer in remission having MRD who are not responsive to the treatment with the immunogenetic composition and display cancer relapse after a first cycle of treatment using the immunogenetic composition.

In certain exemplary embodiment, the method further comprises administering to the identified subject with at least one dose of the immunogenetic composition comprising an effective amount of allogeneic leukemia-derived dendritic cells. In certain exemplary embodiments, the immunogenetic composition is administered to the subject according to a regimen comprising: 4 biweekly prime doses of the immunogenetic composition; and optional one booster dose of the immunogenetic composition applied at week 14 or two booster doses of the immunogenetic composition applied at week 14 and week 18; wherein each biweekly prime dose comprises 25E6 or 50E6 allogeneic leukemia-derived dendritic cells and each booster dose comprises 10E6 allogeneic leukemia-derived dendritic cells.

In various exemplary embodiments, the subject has an elevated baseline level of HLA-DR+CD45RA+ dendritic cells relative to a reference level of the biomarker.

In another aspect, provided here is a method of selecting a subject with cancer in remission having measurable residual disease (MRD) who is likely to have a reduced MRD level or disappearance of the MRD in response to a treatment with an immunogenetic composition, predicting a risk of developing cancer relapse or recurrence, and/or identifying a need of a continuation treatment with the immunogenetic composition to prevent or delay relapse or recurrence of cancer or reduce the risk of developing cancer relapse, or further deepen the MRD response levels or the disappearance of the MRD, the method comprising: assessing a baseline level of a biomarker in peripheral blood of the subject before treating the subject with the immunogenetic composition; assessing one or more post-treatment levels of the biomarker in peripheral blood of the subject after the subject is treated with the immunogenetic composition according to a first dosage regimen; and identifying the subject as being likely to be responsive to the treatment with the immunogenetic composition and as in need of continuous treatment with the immunogenetic composition to prevent or delay relapse or recurrence of cancer, or further deepen the MRD response levels or the disappearance of the MRD, if the one or more post-treatment levels of the biomarker are greater than or less than the baseline level of the biomarker; wherein the immunogenetic composition comprises allogeneic leukemia-derived dendritic cells.

In certain embodiments, the baseline level of the biomarker corresponds to a mean number of the at least one subset of dendritic cells in the peripheral blood of the subject and is assessed by measuring the number of the at least one subset of dendritic cells in at least one blood samples periodically obtained from the subject before the subject is administered with the immunogenetic composition. In some embodiments, the baseline level of the biomarker corresponds to a mean number of the at least one subset of dendritic cells in the peripheral blood of the subject and is assessed by measuring the number of the at least one subset of dendritic cells in at least one blood samples periodically obtained from the subject before the subject is administered with the immunogenetic composition, wherein each of the one or more post-treatment levels of the biomarker corresponds to a mean or average number of the at least one subset of dendritic cells in the peripheral blood of the subject and is assessed by measuring the number of the at least one subset of dendritic cells in a blood sample obtained from the subject after the subject is treated with treated with the immunogenetic composition according to the first dosage regimen.

In certain exemplary embodiments, wherein the one or more post-treatment levels of the biomarker include a first post-treatment level and a second post-treatment level of the biomarker in the peripheral blood of the subject; wherein the first post-treatment level of the biomarker is assessed two days after completion of the first dosage regimen; and wherein the second post-treatment level of the biomarker is assessed two weeks after completion of the first dosage regimen.

In certain exemplary embodiments, the method further comprises identifying the subject as in need of a continuous treatment with the immunogenetic composition for at least one booster regimen if the second post-treatment level of the biomarker is greater than the first post-treatment level of the biomarker.

In various exemplary embodiments, the subject has an elevated baseline level of HLA-DR+CD45RA+ dendritic cells relative to a reference level of the biomarker.

In another aspect, disclosed herein is a method of identifying or selecting a subject with cancer in remission having measurable residual disease (MRD) as being likely to have a reduced MRD level or disappearance of the MRD in response to a treatment with an immunogenetic composition and treating the identified subject to prevent or delay relapse or recurrence of cancer, and/or reduce a risk of relapse or recurrence of cancer, deepen the MRD levels, or the disappearance of the MRD, the method comprising: assessing a baseline level of a biomarker in a peripheral blood of a subject before the subject is treated with the immunogenetic composition in comparison with a predetermined reference level of the biomarker; identifying or selecting the subject as being likely to have a reduced MRD level or disappearance of the MRD in response to the treatment with the immunogenetic composition if the level of baseline level of the biomarker in the peripheral blood of the subject is altered compared to the predetermined reference level of the biomarker; and administering to the identified subject with at least one dose of the immunogenetic composition comprising an effective amount of allogeneic leukemia-derived dendritic cells.

In certain embodiments, the predetermined level of the biomarker corresponds to a mean number of the at least one subset of dendritic cells in the peripheral blood of one or more separate subjects with the cancer in remission having MRD who are not responsive to the treatment with the immunogenetic composition and display cancer relapse after a first cycle of treatment using the immunogenetic composition.

In various exemplary embodiments, the subject has an elevated baseline level of HLA-DR+CD45RA+ dendritic cells relative to a reference level of the biomarker.

In another aspect, disclosed herein is a method of identifying a subject with cancer in remission having measurable residual disease (MRD) who has been treated with an immunogenetic composition according to a first dosage regimen and is likely in need of continuous or booster treatment with the immunogenetic composition, and treating the subject to prevent or delay relapse or recurrence of cancer, and/or reduce a risk of relapse or recurrence of cancer, the method comprising: assessing a baseline level of a biomarker in peripheral blood of the subject before the subject is treated with the immunogenetic composition; assessing one or more post-treatment levels of the biomarker in peripheral blood of the subject at one or more time points after the subject is treated with the immunogenetic composition according to a first dosage regimen; identifying the subject as being likely to have a reduced MRD level or disappearance of the MRD in response to the treatment with the immunogenetic composition and as in need of continuous treatment with the immunogenetic composition to prevent or delay relapse or recurrence of cancer if the one or more post-treatment levels of the biomarker are greater than the baseline level of the biomarker level; and administering to the identified subject with the immunogenetic composition according to a second booster regimen; wherein the immunogenetic composition comprises allogeneic leukemia-derived dendritic cells.

In certain exemplary embodiments, the baseline level of the biomarker corresponds to a mean number of the at least one subset of dendritic cells in the peripheral blood of the subject and is assessed by measuring the number of the at least one subset of dendritic cells in at least one blood samples periodically obtained from the subject before the subject is administered with the immunogenetic composition; and the one or more post-treatment levels of the biomarker corresponds to a mean or average number of the at least one subset of dendritic cells in the peripheral blood of the subject and is assessed by measuring the number of the at least one subset of dendritic cells in a blood sample obtained from the subject after the subject is treated with the immunogenetic composition according to the first dosage regimen.

In some exemplary embodiment, the one or more post-treatment levels of the biomarker include a first post-treatment level and a second post-treatment level of the biomarker in the peripheral blood of the subject; wherein the first post-treatment level of the biomarker is assessed two days after completion of the first dosage regimen; and wherein the second post-treatment level of the biomarker is assessed two weeks after completion of the first dosage regimen.

In certain embodiments, the method further comprises identifying the subject as in need of a continuous treatment with the immunogenetic composition for at least one booster regimen if the second post-treatment level of the biomarker is greater than the first post-treatment level of the biomarker.

In certain embodiments, the method the first dosage regimen includes 4 biweekly doses of the immunogenetic composition and two or more booster doses of the immunogenetic composition applied once every 4 weeks thereafter, for example, at weeks 10, 14, week 18, wherein each biweekly dose comprises 25E6 or 50E6 allogeneic leukemia-derived dendritic cells, and wherein each booster dose comprises 10E6 allogeneic leukemia-derived dendritic cells. In certain embodiments, the second booster regimen comprises at least one booster dose of the immunogenetic composition applied after week 18. In certain embodiments, each booster dose comprises 10E6 allogeneic leukemia-derived dendritic cells.

In certain exemplary embodiments, the method of any one of claims 62-67, wherein each booster dose in the second booster regimen is applied to the subject once every 4 weeks.

In certain exemplary embodiments, the subject is treated with the immunogenetic composition for at least one booster dose until the subject shows MRD conversion or disappearance.

In certain exemplary embodiments, the immunogenetic composition is applied to the subject via an intradermal route. In certain embodiments, the immunogenetic composition is applied to the subject via injection. In some embodiments, the immunogenetic composition is applied to the subject via intradermal injection.

In various exemplary embodiments, the biomarker used in the above various embodiments is chosen from dendritic cells, wherein the biomarker is chosen from dendritic cells, CD8+ T cells, B cells, NK cells, CD4 CD161+ T cells, CD14+CD16− non-inflammatory monocytes, or a combination thereof. In certain exemplary embodiments, the dendritic cells may be conventional dendritic cells DC1 (cDC1), conventional DC2 (cDC2), plasmacytoid dendritic cells (pDC), or a combination thereof. In some embodiments, the dendritic cells may be CD141+/CLEC9A+ cDC1, cDC2, CD163+ cDC2, HLA-DR+/CD123+ pDC cells, or combination thereof. In some embodiments, the NK cells may be CD56++NK cells and/or CD56+NK cells. In some embodiments, the CD8+ T cells comprise CD8+/CD45RA+ T cells, CD8+/CD45RA−/CCR7+CM T cells, and/or CD8/RO+ T cells.

In some embodiments, the cancer in the above various embodiments is a liquid cancer. In some further embodiments, the liquid cancer is selected from the group consisting of leukemia, lymphoma, and myeloma. In certain embodiments, the liquid cancer is acute myeloid leukemia (AML).

In some embodiments, the immunogenetic composition used in the above embodiments further comprises a pharmaceutically acceptable carrier, adjuvants, excipients, and/or diluents.

In various exemplary embodiments, the allogeneic leukemia-derived cell used in the above various embodiments is CD34-positive, CD1a-positive, and CD83-positive. In certain exemplary embodiments, the allogeneic leukemia-derived cell expresses a cell surface marker selected from the group consisting of DC-SIGN, Langerin, CD80, CD86, CD70, CD40, and any combination thereof. In certain embodiments, the allogeneic leukemia-derived cell is CD34-positive, CD1a-positive, CD83-positive, CD80-positive, CD86-positive, and CD40-positive. In some other embodiments, the allogeneic leukemia-derived cell may be CD14-negative. In certain exemplary embodiments, the allogeneic leukemia-derived cell is derived from the DCOne cell line.

In various exemplary embodiments, the allogeneic leukemia-derived cell used in the above various embodiments comprise a tumor associated antigen or a nucleic acid encoding a tumor associated antigen that is associated with a cancer cell in the subject. In certain embodiments, the tumor associated antigen is chosen from WT-1, MUC-1, RHAMM, PRAME, p53, Survivin, or combinations thereof. In certain embodiments, the allogeneic leukemia-derived cells comprise at least one tumor associated antigen or a nucleic acid encoding at least one tumor associated antigen that is not associated with a cancer cell in the subject. In certain embodiments, the allogeneic leukemia-derived cells have been inactivated, optionally, via irradiation.

In various exemplary embodiments, the subject in the above various embodiments receives one or more biweekly doses of about 25e6 or about 50e6 allogeneic leukemia-derived dendritic cells. In certain exemplary embodiments, the subject receives one or more booster doses, each dose comprising about 10e6 allogeneic leukemia-derived dendritic cells.

In certain exemplary embodiments, each booster dose is administered to the subject about 28 days after a previous dose.

In various exemplary embodiments, the subject achieves MRD conversion or disappearance after receiving treatment with the composition comprising the allogeneic leukemia-derived dendritic cells.

In certain embodiments, the composition used in the above various embodiment is administered to the subject by injection. In various exemplary embodiments, the subject may have previously been treated with allogeneic leukemia-derived dendritic cells. In certain exemplary embodiments, the subject is incomplete remission (CR).

In the above various exemplary embodiments, the cancer may be chosen from acute myeloid leukemia, myelodysplastic syndrome (MDS) multiple myeloma (MM), or a combination thereof. In some embodiments, the cancer may be acute myeloid leukemia (AML). In certain situations, the subject is ineligible for accepting hematopoietic stem cell transplantation. In certain situations, the subject has been initially treated with a chemotherapy and/or debulking surgery, and the remission is induced by the chemotherapy and/or the debulking surgery.

In the above various exemplary embodiments, the subject may be a non-human animal. In certain exemplary embodiments, the subject is a human.

### BRIEF DESCRIPTION OF THE DRAWINGS

The foregoing and other features and advantages of the present disclosure will be more fully understood from the following detailed description of illustrative embodiments taken in conjunction with the accompanying drawings.

FIGS. 1A-1D depict graphs illustrating the higher number and durable vaccine induced functional T cell responses measured using IFNγ ELISpot assay, according to an embodiment of the present disclosure. FIG. 1A illustrates the total immune response induced by the cancer vaccine comprising DCP-001 according to one embodiments of the present disclosure, assessed by IFNγ ELISPOT assay; FIG. 1B illustrates sustained ELISPOT responses at least 2 timepoints post first dose in patients with relapse, patients with stable MRD, and MRD responders (either at least a 10-fold decrease in MRD level or a full MRD conversion/disappearance). FIG. 1C illustrates vaccine induced responses to tumor associated antigens (either WT1, Prame or Rhamm) in patients with or without relapse during the first 32 weeks of the study. FIG. 1D is a graph illustrating ELISPOT responses for either of the three antigens, during vaccination with DCP-001 (indicated vaccination timepoints by arrows).

FIGS. 1E-1H depict graphs illustrating the relapse free survival and overall survival of patients after the vaccination with the DCP-001. FIG. 1E is a graph illustrating the results of relapse free survival of the patients up to 48 months post the 1^{st} dose of the DCP-001. FIG. 1F illustrates the relapse free survival by MRD conversion up to 48 months post the 1^{st} dose of the DCP-001. FIG. 1G is a graph illustrating the results of overall survival (OS) of the patients up to 48 months post the 1^{st} dose of the DCP-001. FIG. 1H illustrates the overall survival by MRD conversion up to 48 months post the 1^{st} dose of the DCP-001.

FIGS. 2A-2D depict graphs illustrating the baseline levels of CD8 T cells of patients who relapsed compared to patients who did not relapse, according to one embodiment of the present disclosure. FIG. 2A illustrates the baseline levels of the population of CD8+T memory cells (CM) without leukocyte common antigen isoform CD45RA and with the chemokine receptor CCR7 (CD8+, CD45RA−, CCR7+, CM) in patients who relapsed and patients who did not relapse, measured in 15 patients according to one embodiment of the present disclosure. FIG. 2B illustrates the baseline levels of the population of CD8+, CD45RA−, CCR7+, CM T cells in patients who relapsed and patients who did not relapse, measured in 20 patients according to one embodiment of the present disclosure. FIG. 2C illustrates the baseline levels of the population of CD8 RA+ T cells in patients who relapsed compared to patients who did not relapse. FIG. 2D illustrates the baseline levels of the population of CD8+RO+ Cells (EM and CM) in patients who relapsed, remained in CR with MRD, and an MRD response, respectively, according to one embodiment of the present disclosure.

FIG. 3 depicts graphs illustrating the baseline levels of B cells in patients who relapsed compared to patients who did not relapse according to one embodiment of the present disclosure.

FIG. 4 depicts graphs illustrating the baseline levels of cDC2 (HLA-DR+, CD14−, CD11b−, CD11C+) cells in patients who relapsed compared to patients who did not relapse, according to one embodiment of the present disclosure.

FIG. 5 depicts graphs illustrating the baseline levels of NK cells (CD56+) in patients who relapsed compared to patients who did not relapse, according to one embodiment of the present disclosure.

FIG. 6 depicts graphs illustrating the baseline levels of CD4 CD161+ T cells in patients who relapsed, patients who remained in CR with MRD, and patients with an MRD response, according to one embodiment of the present disclosure.

FIGS. 7A-7D depict graphs illustrating the status of dendritic cells cDC1 (HLA-DR+, CD14−, CD11B−, CD11c+) in blood samples collected from the patients before, during, and after the course of the vaccination with DCP-001, where FIG. 7A illustrates the status of the cDC1 cells in patients who relapsed, FIG. 7B illustrates the status of the cDC1 cells in patients who remained in complete remission with stable MRD, FIG. 7C illustrates the status of the cDC1 cells in patients who had an MRD response, and FIG. 7D illustrates significance analysis of the level of cDC1 (HLA-DR+, CD14−, CD11B−, CD11c+) in the blood of the patients before, during, and after the course of the vaccination with DCP-001.

FIGS. 8A-8D depict graphs illustrating the status of dendritic cells cDC2 (HLA-DR+CD14−, CD11b−, CD11c+) in blood samples collected from the patients before, during, and after the course of the vaccination with DCP-001, where FIG. 8A illustrates the status of the cDC2 cells in patients who relapsed, FIG. 8B illustrates the status of the cDC2 cells in patients who remained in complete remission with stable MRD, FIG. 8C illustrates the status of the cDC2 cells in patients who had MRD response, and FIG. 8D illustrates the level of cDC2 (HLA-DR+, CD14−, CD11B−, CD11c+) in the blood of the patients before, during, and after the course of the vaccination with DCP-001.

FIGS. 9A and 9B depict graphs illustrating the status of dendritic cells CD163+ cDC2 (HLA-DR+, CD14−, CD11b−, CD1c+, and CD163+) in blood samples collected from the patients before, during, and after the vaccination course with DCP-001.

FIGS. 10A and 10B depict graphs illustrating the analysis of the change of level of CD14+CD16− non-inflammatory monocytes in blood samples collected from the patients before, during, and after the course of the vaccination with DCP-001, according to one embodiment of the present disclosure.

FIGS. 11A-11C depict graphs illustrating the status of CD56++NK in blood samples collected from the patients before, during, and after the course of the vaccination with DCP-001, where FIG. 11A illustrates the status of the CD56++NK cells in patients who relapsed, FIG. 11B illustrates the status of the CD56++NK cells in patients who remained in complete remission with stable MRD, FIG. 11C illustrates the status of the CD56++NK cells in patients who had an MRD response.

FIGS. 11D and 11E illustrate the level of CD56++NK in the blood of the patients before, during, and after the course of the vaccination with DCP-001.

FIG. 12A illustrates the level of CD56+NK cells in the blood of the patients before, during, and after the course of the vaccination with DCP-001.

FIGS. 12B-12D depict graphs illustrating the status of CD56+NK cells in blood samples collected from the patients before, during, and after the course of the vaccination with DCP-001, as described in Example 1, where FIG. 12B illustrates the status of the CD56+NK cells in patients who relapsed, FIG. 12C illustrates the status of the CD56+NK cells in patients who remained in complete remission with stable MRD, and FIG. 12D illustrates the status of the CD56+NK cells in patients who had an MRD response.

FIG. 13A illustrates analysis of the level of B cells in the blood of the patients before, during, and after the course of the vaccination with DCP-001.

FIGS. 13B-13D depict graphs illustrating the status of B cells in blood samples collected from the patients before, during, and after the course of the vaccination with DCP-001, where FIG. 13B illustrates the status of the B cells in patients who relapsed, FIG. 13C illustrates the status of B cells in patients who remained in complete remission with stable MRD, and FIG. 13D illustrates the status of B cells in patients who had MRD response. FIGS. 14A and 14B illustrate the level of HLA-DR+, CD11 b+, CD14+, CD206−, CD163+ cells in the blood of the patients before, during, and after the course of the vaccination with DCP-001.

FIG. 15 illustrates the level of CD16-CD56+NK cells in the blood of the patients before, during, and after the course of the vaccination with DCP-001.

FIG. 16 illustrates the level of CD4+ LAG3+ cells in the blood of the patients before, during, and after the course of the vaccination with DCP-001.

FIG. 17A-17C depicts a Uniform Manifold Approximation and Projection (UMAP) plot of CD45, CD3−, CD19−, CD56−, CD14−, CD15−, CD16−, CD11b−, HLA-DR+ lymphocytes. FIG. 17A and FIG. 17B show the expression levels of the indicated antigens, showing no difference in expression levels, but a lower level of CD45RA+/DR+ cells in the relapse patients as indicated on the overlay plot (FIG. 17C), where the blue shows the patients in complete remission with a dominance of these cells at baseline.

### DETAILED DESCRIPTION

Methods for enhancing the effect of immune cells (e.g., genetically modified immune cells) in vivo are provided. In particular, methods of treating a disease or disorder are provided in which an inactivated modified cell of leukemic origin is administered to a subject who has undergone adoptive cell therapy with said modified immune. Such methods may prolong the duration of the clinical effect of a genetically modified immune cell, and/or function to stabilize subjects following adoptive cell therapy. In certain embodiments, the modified cell of leukemic origin is inactivated (e.g., via irradiation). In certain embodiments, the inactivated modified cell of leukemic origin is an irradiated DCOne-derived cell.

It is to be understood that the methods described herein are not limited to particular methods and experimental conditions disclosed herein as such methods and conditions may vary. It is also to be understood that the terminology used herein is for the purpose of describing particular embodiments only, and is not intended to be limiting. The methods described herein use conventional molecular and cellular biological and immunological techniques that are well within the skill of the ordinary artisan. Such techniques are well known to the skilled artisan and are explained in the scientific literature.

### A. DEFINITIONS

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element, or at least one element.

It should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. For instance, “about” as used herein when referring to a measurable value such as an amount, a temporal duration, and the like, is meant to encompass variations of ±20% or ±10%, e.g., ±5%, e.g., 1%, and e.g., ±0.1% from the specified value, as such variations are appropriate to perform the disclosed methods.

As used herein, the term “adjuvant” and the term “immuno-adjuvant” refer to components in vaccines or therapeutic compositions that enhance the production of antibodies without acting as antigens themselves. An adjuvant may be a compound added in a vaccine or therapeutic composition that increases the specific immune response against a co-inoculated antigen. Adjuvants may be added to a vaccine to modify the immune response by boosting it such as to give a higher amount of antibodies and a longer lasting protection, thus minimizing the amount of injected foreign material. Adjuvants may also be used to enhance the efficacy of vaccine by helping to subvert the immune response to particular cells type of immune system, for example by activating the T cells instead of antibody-secreting B cells depending on the type of the vaccine. Adjuvants are also used in the production of antibodies from immunized animals. Adjuvants are well known to those of skill in the art. Traditional vaccine usually needs an adjuvant. Although adjuvants have traditionally been viewed as substances that aid the immune response to antigen, adjuvants have also evolved as substances that can aid in stabilizing formulations of antigens, especially for vaccines administered for animal health.

As used herein, the phase “administration of a vaccine” refers to introduce a vaccine into a body of an animal or a human being. As is understood by an ordinary skilled person, it can be done in a variety of manners. For example, administration of a vaccine may be done intramuscularly, subcutaneously, intravenously, intranasally, intradermally, intrabursally, in ovo, ocularly, orally, intra-tracheally or intra-bronchially, as well as combinations of such modalities. The dose of the vaccine may vary with the size of the intended vaccination subject.

As used herein, the term “antibody” refers to a protein produced by plasma cells that are used by an immune system to identify and neutralize foreign objects, for example, bacteria and viruses. An “antibody” is also known as an “immunoglobulin.” Each antibody recognizes a specific part of a specific foreign object, called an antigen, and binds the specific antigen. Antibodies can cause agglutination and precipitation of antibody-antigen products, prime for phagocytosis by macrophages and other cells, block viral receptors, and stimulate other immune responses, such as the complement pathway.

As used herein, to “alleviate” a disease means reducing the severity of one or more symptoms of the disease.

As used here, the term “animal” refers to humans as well as non-human animals. In an exemplary embodiment, the non-human animal is a mammal (e.g., a rodent, a mouse, a rat, a rabbit, a monkey, a dog, a cat, a primate, or a pig). An animal may be a domesticated animal. An animal may be a transgenic animal.

As used herein, the term “autologous” is meant to refer to any material derived from the same individual to which it is later to be re-introduced into the individual. As used herein, the term “allogeneic” refers to the involvement of living tissues or cells that are genetically dissimilar and hence immunologically incompatible, with respect to a subject in need of treatment. While genetically dissimilar, an allogeneic cell, e.g., an allogeneic leukemia-derived cell described herein, is derived from the same species. For example, a method described herein comprising administering to a subject an allogeneic leukemia-derived cell, refers to the administration of a leukemia-derived cell that is genetically dissimilar to the subject, albeit still of the same species.

As used herein, the term “comprising,” the term “having,” the term “including,” and variations of these words are intended to be open-ended and mean that there may be additional elements other than the listed elements.

As used herein, the term “cross-priming” refers to the activation of naïve CD8+ T cells by patients own antigen-presenting cells such as dendritic cells cDC1, cDC2, pDC, or a combination thereof.

As used herein, a “disease” refers to a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “dosage” refers to the administering of a specific amount, number, and frequency of doses over a specified period of time. Dosage implies duration. A “dosage regimen” is a treatment plan for administering a drug over a period of time.

As used herein, the term “dosage form,” the term “form,” and the term “unit dose” refer to a method of preparing pharmaceutical products in which individual doses of medications are prepared and delivered. Dosage forms typically involve a mixture of active drug components and nondrug components (excipients), along with other non-reusable material that may not be considered either ingredient or packaging.

As used herein, the term “dose” refers to a specified amount of medication taken at one time.

As used herein, the term “drug” refers to a material that may have a biological effect on a cell, including but not limited to small organic molecules, inorganic compounds, polymers such as nucleic acids, peptides, saccharides, or other biologic materials, nanoparticles, etc.

As used herein, “effective amount,” a “therapeutically effective amount,” an “effective dose,” or grammatical variations thereof interchangeably refers to an amount of an agent, a compound, a formulation, a material, a cell, or a composition, as described herein effective to achieve a particular biological result or provides a therapeutic or prophylactic benefit. Such results may include, but are not limited to, an amount that when administered to a mammal, causes a detectable level of immune suppression or tolerance compared to the immune response detected in the absence of the composition of the disclosure. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like. In certain situation, a “therapeutically effective amount” is used to mean an amount sufficient to prevent, correct and/or normalize an abnormal physiological response or a measurable improvement in a desirable response (e.g., enhanced adaptive immune response).

As used herein, “endogenous” refers to any material from or produced inside an organism, cell, tissue, or system.

As used herein, the term “excipient” refers to a natural or synthetic substance formulated alongside the active ingredient of a medication, included for the purpose of bulking-up formulations that contain potent active ingredients (thus often referred to as “bulking agents,” “fillers,” or “diluents”), or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. The selection of appropriate excipients also depends upon the route of administration and the dosage form, as well as the active ingredient and other factors. Though excipients were at one time considered to be “inactive” ingredients, they are now understood to be “a key determinant of dosage form performance.” For purposes of the present invention, the term “expression” and the term “gene expression” refer to a process by which information from a gene or a fragment of DNA is used in the synthesis of a functional gene product. A gene which encodes a protein will, when expressed, be transcribed and translated to produce that protein.

As used herein, the term “expression” refers to a process by which information from a gene or a fragment of DNA is gene expression.

As used herein, the term “gene therapy” refers to the purposeful delivery of genetic material to cells for the purpose of treating disease or biomedical investigation and research. Gene therapy includes the delivery of a polynucleotide to a cell to express an exogenous nucleotide sequence, to inhibit, eliminate, augment, or alter expression of an endogenous nucleotide sequence, or to produce a specific physiological characteristic not naturally associated with the cell. In some cases, the polynucleotide itself, when delivered to a cell, can alter expression of a gene in the cell.

As used herein, the term “immunogen” and the term “immunogenic composition” interchangeably refer to a substance or material (including antigens) that is able to induce a specific immune response against the immunogen in a subject who is in need of an immune response against said immunogen. The immunogenic composition may include an adjuvant and optionally one or more pharmaceutically acceptable carriers, excipients and/or diluents. Both natural and synthetic substances may be immunogens. In various embodiments, the immunogenic composition comprises an allogeneic leukemia-derived cell.

As used herein, the term “immunogenicity” refers to the ability to of a particular substance, such as an antigen or epitope, to provoke an immune response in the body of a human or animal. In other words, immunogenicity is the ability to induce a humoral and/or cell-mediated immune responses.

As used herein, the term “immunization dose” refers to the amount of antigen or immunogen needed to precipitate an immune response. This amount will vary with the presence and effectiveness of various adjuvants. This amount will vary with the animal and the antigen, immunogen and/or adjuvant. The immunization dose is easily determined by methods well known to those skilled in the art, such as by conducting statistically valid host animal immunization and challenge studies.

As used herein, the term “immune response” refers to any response to an immunogen, for example, an antigen or antigenic determinant, by the immune system of a subject (e.g., a human). Exemplary immune responses include humoral immune responses (e.g., production of antigen-specific antibodies, e.g., neutralizing antibodies (NAbs)) and cell-mediated immune responses (e.g., lymphocyte proliferation).

As used herein, the term “individual” refers to an individual mammal, such as a human being.

As used herein, the term “intra-peritoneal injection” or the term “IP injection” refer to the injection of a substance into the peritoneum.

As used herein, the term “medical therapy” refers to prophylactic, diagnostic and therapeutic regimens carried out in vivo or ex vivo on humans or other mammals.

As used herein, the term “parenteral route” or “parenteral administration” refers to the administration of a composition, such as a drug in a manner other than through the digestive tract. Parenteral routes include routes such as subcutaneous (s.c.), intravenous (i.v.), intramuscular (i.m.), intra-arterial, transdermal, intranasal, sub-lingual and intraosseous, etc., or infusion techniques. For example, intravenous is also known as I.V., which is giving directly into a vein with injection. As the drug directly goes into the systemic circulation, it reaches the site of action resulting in the onset the action.

As used herein, the term “pharmaceutically acceptable” refers to a compound or drug approved or approvable by a regulatory agency of a federal or a state government, listed or listable in the U.S. Pharmacopeia or in other generally recognized pharmacopeia for use in mammals, including humans. For example, a “pharmaceutically acceptable diluent, excipient, carrier, or adjuvant” is a diluent, excipient, carrier, or adjuvant which is physiologically acceptable to the subject while retaining the therapeutic properties of the pharmaceutical composition with which it is administered. One exemplary pharmaceutically acceptable carrier is physiological saline.

As used herein, the term “pharmaceutically acceptable carrier” refers to any carrier that does not itself induce the production of antibodies harmful to an individual or a subject receiving a composition. For example, pharmaceutically acceptable carriers may be large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immuno-stimulating agents (“adjuvants”).

As used herein, the term “pharmaceutically acceptable salt” refers to those salts of compounds that are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. They may be prepared in situ when finally isolating and purifying the compounds of the invention, or separately by reacting them with pharmaceutically acceptable non-toxic bases or acids, including inorganic or organic bases and inorganic or organic acids. Pharmaceutically acceptable salts may be obtained using standard procedures well known in the art, for example by mixing a compound of the present invention with a suitable acid, for instance an inorganic acid or an organic acid. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts.

As used herein, the term “pharmaceutical composition” refers to a product comprising one or more active ingredients, and one or more other components such as carriers, stabilizers, diluents, dispersing agents, suspending agents, thickening agents, and/or excipients, etc. A pharmaceutical composition includes enough of the active object compound to produce the desired effect upon the progress or condition of diseases and facilitates the administration of the active ingredients to an organism. Multiple techniques of administering the active ingredients exist in the art including, but not limited to, topical, ophthalmic, intraocular, periocular, intravenous, oral, aerosol, parenteral, and administration. By “pharmaceutically acceptable,” it is meant the carrier, diluent or excipient must be compatible with the other ingredients of the formulation and not deleterious to the recipient thereof, i.e., the subject.

As used herein, the term “pharmaceutical formulation” and the term “drug formulation” refer to a mixtures or a structure in which different chemical substances, including the active drug, are combined to form a final medicinal product, such as a sterile product, a capsule, a tablet, a powder, a granule, a solution, an emulsion, a topical preparation, a non-conventional product such as semi-solid or sustained-release preparations, liquid, etc. Pharmaceutical formulation is prepared according to a specific procedure, a “formula.” The drug formed varies by the route of administration. For example, oral drugs are normally taken as tablet or capsules.

As used herein, the term “prevent” refers to stop from happening or to make something not happen.

As used herein, the term “prognostic analysis” or “prognostic diagnosis” refers to an analysis or diagnosis that estimate or predict the course of a disease or the outcome of a clinical treatment of the disease.

As used herein, the term “prognostic composite biomarker” or refers to a panel of biological parameters that aids the prediction or prognostic analysis of the presence or severity of a disease or condition of interest, or identifies an individual or a subject as be in a condition of whether or not likely to have a MRD response to a treatment or continuous treatment, and/or success of the treatment.

As used herein, the term “recurrence” or “relapse” of cancer refers to a situation that cancer is found after treatment and after a period of time when the cancer could not be detected. A recurrent cancer may come back in the same place it first started or might come back somewhere else in the body. When cancer spreads to a new part of the body, it is still named after the part of the body where it started. There are different types of cancer recurrence: local recurrence means that the cancer has come back in the same place it first started; regional recurrence means that the cancer has come back in the lymph nodes near the place it first started; distant recurrence means the cancer has come back in another part of the body, some distance from where it started (often the lungs, liver, bone, or brain).

As used herein, the term “relapse-free survival” refers to a subject survives from a cancer without any signs or symptoms of that cancer after a primary treatment for the cancer ends. It is also called DFS, disease-free survival, and RFS, recurrence-free survival.

As used herein, the term “stimulate,” the term “immuno-stimulate” refers to induce the activation or increase the activity of any components in an immune system. For example, T cell activation requires at least two signals to become fully activated. The first occurs after engagement of the T cell antigen-specific receptor (TCR) by the antigen-major histocompatibility complex (MHC), and the second by subsequent engagement of co-stimulatory molecules. Once stimulated, the T cells will recognize the antigen or vaccine used during stimulation or activation of the T cells.

As used herein, the terms “subject” or “individual” or “patient,” are used interchangeably herein, and refers to any subject, particularly a mammalian subject, for whom diagnosis or therapy is desired. Mammalian subjects include for example, humans, domestic animals, farm animals, and zoo, sports, or pet animals such as dogs, cats, guinea pigs, rabbits, rats, mice, horses, cattle, and cows.

As used herein, the term “target” refers to a living organism or a biological molecule to which some other entity, like a ligand or a drug, is directed and/or binds. For example, “target protein” may a biological molecule, such as a protein or protein complex, a receptor, or a portion of a biological molecule, etc., capable of being bound and regulated by a biologically active composition such as a pharmacologically active drug compound.

As used herein, the term “therapeutic” as used herein means a treatment and/or prophylaxis. A therapeutic effect is obtained by suppression, remission, or eradication of a disease state.

As used herein, the terms “treat” or “treatment” refer to both therapeutic treatment and prophylactic or preventative measures, wherein the object is to prevent or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to or at risk of having the condition or disorder or those in which the condition or disorder is to be prevented. In certain embodiments, treatment also refers to preventing recurrence and delaying recurrence of a disease or disorder, e.g., a liquid cancer.

As used herein, the term “cancer vaccine” refers to a biological compound or an agent including particular cells, or a composition comprising said biological compound or agent used as a treatment vaccine or therapeutic vaccine to treat existing cancer, and/or to reduce or eliminate existing cancer cells. In some embodiments, the cancer vaccine may be used for preventing or delaying relapse of the cancer, destroy any cancer cells remaining in the body of the subject after an initial treatment, and/or stop from the cancer cells from growing or spreading. In some embodiment, the cancer vaccine is injected into a human or animal body, which stimulates the body's immune system to recognize the agent as foreign, destroy it, and keep a record of it.

RANGES: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. ALLOGENEIC LEUKEMIA-DERIVED CELLS

As used herein, the term “leukemia-derived cell” refers to a cell of leukemic origin that is capable of presenting an antigen, or an immunogenic portion thereof, together with an MHC class I complex or MHC class II complex. The term “allogenceic leukemia-derived cell” refers to a leukemia-derived cell that is genetically dissimilar with respect to the subject it is utilized to treat, yet is of the same species. In some embodiments, an allogeneic leukemia-derived cell provided herein comprises a dendritic cell phenotype. In some embodiments, an allogeneic leukemia-derived cell provided herein comprises a mature dendritic cell phenotype. The term “dendritic cell,” as used herein, refers to a professional antigen presenting cell (APC) that can take up an antigen, and is capable of presenting the antigen, or an immunogenic portion thereof, together with an MHC class I complex or MHC class II complex. In some embodiments, an allogeneic leukemia-derived cell as described herein has a mature dendritic cell phenotype capable of performing similar functions to those of a mature dendritic cell. The term dendritic cell includes both immature dendritic cells (“imDC”) and mature dendritic cells (“mDC”), depending on maturity. In certain embodiments, the allogeneic leukemia-derived cell is a cell derived from cell line DCOne as deposited under the conditions of the Budapest treaty with the DSMZ under accession number DSMZ ACC3189 on 15 Nov. 2012. The process of obtaining mature cells from the deposited DCOne cell line is for instance described in EP2931878B1, the disclosure of which is incorporated by reference herein in its entirety.

In certain embodiments, the allogeneic leukemia-derived cell is derived from a leukemia cell. In certain embodiments, the allogeneic leukemia-derived cell is derived from a subject having leukemia (e.g., a genetically dissimilar subject with respect to the subject that the leukemia-derived cell is utilized to treat). In certain embodiments, the allogeneic leukemia-derived cell is derived from the peripheral blood of a patient having leukemia. In certain embodiments, the allogeneic leukemia-derived cell is derived from the peripheral blood of a patient having acute myeloid leukemia. The skilled artisan will recognize that an allogeneic leukemia-derived cell can be derived from any patient-obtained peripheral blood, wherein the patient has any type of leukemia, given that the leukemia-derived cell thus derived comprises the characteristics disclosed herein.

In certain embodiments, the allogeneic leukemia-derived cell is CD34-positive, CD1a-positive, and CD83-positive. In certain embodiments, the allogeneic leukemia-derived cell comprises a cell surface marker selected from the group consisting of CD14, DC-SIGN, Langerin, CD40, CD70, CD80, CD83, CD86, and any combination thereof. In certain embodiments, the allogeneic leukemia-derived cell expresses a cell surface marker selected from the group consisting of CD14, DC-SIGN, Langerin, CD40, CD70, CD80, CD83, CD86, and any combination thereof. In certain embodiments, the allogeneic leukemia-derived cell comprises an MHC class I molecule. In certain embodiments, the allogeneic leukemia-derived cell comprises an MHC class II molecule.

In certain embodiments, the allogeneic leukemia-derived cell comprises a genetic aberration between chromosome 11p15.5 to 11p12. In certain embodiments, the genetic aberration encompasses about 16 Mb of genomic regions (e.g., from about 20.7 Mb to about 36.6 Mb). In certain embodiments, the genetic aberration contains a loss of about 60 known and unknown genes.

In certain embodiments, the allogeneic leukemia-derived cell comprises a co-stimulatory molecule. In certain embodiments, the co-stimulatory molecule includes, without limitation, an MHC class I molecule, BTLA and Toll ligand receptor. Examples of co-stimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

In certain embodiments, the allogeneic leukemia-derived cell comprises at least one endogenous antigen. Depending on the leukemic origin of the leukemia-derived cell, the leukemia-derived cell may comprise at least one known endogenous antigen that is specific to the leukemic origin. In certain embodiments, the endogenous antigen is a tumor-associated antigen. In certain embodiments, the endogenous tumor-associated antigen may be selected from the group consisting of WT-1, RHAMM, PRAME, p53, Survivin, and MUC-1.

In certain embodiments, the allogeneic leukemia-derived cell of the present disclosure is a cell of cell line DCOne as described in PCT Publication Nos. WO 2014/006058 and WO 2014/090795, the disclosures of which are incorporated by reference herein in their entireties. In certain embodiments, an allogeneic leukemia-derived cell of the present disclosure is a cell of cell line DCOne and comprises a mature dendritic cell phenotype that is CD34-positive, CD1a-positive, and CD83-positive. In certain embodiments, the allogeneic leukemia-derived cell is a cell of cell line DCOne and is CD34-positive, CD1a-positive, and CD83-positive. In certain embodiments, the allogeneic leukemia-derived cell is a cell of cell line DCOne and comprises a cell surface marker selected from the group consisting of CD14, DC-SIGN, Langerin, CD80, CD86, CD40, CD70, and any combination thereof. In certain embodiments, the allogeneic leukemia-derived cell is a cell of cell line DCOne and comprises MHC class I. In certain embodiments, the allogeneic leukemia-derived cell is a cell of cell line DCOne and comprises MHC class II. In certain embodiments, the allogeneic leukemia-derived cell is a cell of cell line DCOne and comprises a genetic aberration between chromosome 11p15.5 to 11p12. In certain embodiments, the allogeneic leukemia-derived cell is a cell of cell line DCOne and comprises a genetic aberration that encompasses about 16 Mb of genomic regions (e.g., from about 20.7 Mb to about 36.6 Mb). In certain embodiments, the allogeneic leukemia-derived cell is a cell of cell line DCOne and comprises a genetic aberration that contains a loss of about 60 known and unknown genes.

As provided herein, certain methods utilize the use of an allogeneic leukemia-derived cell, wherein the allogeneic leukemia-derived cell is inactivated. Various methods of inactivating an allogeneic leukemia-derived cell of the present disclosure are known to those of skill in the art. In certain embodiments, the allogeneic leukemia-derived cell is irradiated. In certain embodiments, the allogeneic leukemia-derived cell is irradiated prior to its use in a method disclosed herein. Irradiation can, for example, be achieved by gamma irradiation at 30-150 Gy, e.g., 100 Gy, for a period of 1 to 3 hours, using a standard irradiation device (Gammacell or equivalent). Irradiation ensures that any remaining progenitor cell in a composition comprising the allogeneic leukemia-derived cell, e.g., a CD34 positive cell, cannot continue dividing. The cells may, for example, be irradiated prior to injection into patients, when used as a vaccine, or immediately after cultivating is stopped.

### C. METHODS OF TREATMENT

In one aspect, provided here is a method of treating cancer in a subject in need thereof, comprising administering to a subject having measurable residual disease (MRD) and an altered level of a biomarker in peripheral blood of the subject relative to a reference level of the biomarker, a composition comprising an allogeneic leukemia-derived cell, wherein the biomarker may be chosen from dendritic cells, CD8+CD45RA+ cells, B cells, NK cells, CD4 CD161+ T cells, CD14+CD16− non-inflammatory monocytes, or a combination thereof.

In various exemplary embodiments, the dendritic cell biomarker may be conventional dendritic cells DC1 (cDC1), conventional DC2 (cDC2), plasmacytoid dendritic cells (pDC), or a combination thereof. In some embodiments, the dendritic cells may be CD141+/CLEC9A+ cDC1, cDC2, CD163+ cDC2, HLA-DR+/CD123+ pDC cells, or combination thereof. In some embodiments, the NK cells may be CD56++NK cells and/or CD56+NK cells.

In some embodiments, the cancer is a liquid cancer. In some further embodiments, the liquid cancer is selected from the group consisting of leukemia, lymphoma, myelodysplastic syndrome (MDS) and myeloma. In certain embodiments, the liquid cancer is acute myeloid leukemia (AML).

In various exemplary embodiments the allogeneic leukemia-derived cell comprises WT-1, MUC-1, PRAME, RHAMM, P53, and Survivin.

In various exemplary embodiments, the allogeneic leukemia-derived cell comprises a dendritic cell phenotype. In some embodiments, the allogeneic leukemia-derived cell comprises a mature dendritic cell phenotype. In certain exemplary embodiments, the allogeneic leukemia-derived cell comprises a genetic aberration between chromosome 11p15.5 to 11p12. In certain exemplary embodiments, the genetic aberration encompasses about 16 Mb of genomic regions.

In various exemplary embodiments, the allogeneic leukemia-derived cell is CD34-positive, CD1a-positive, and CD83-positive. In certain exemplary embodiments, the allogeneic leukemia-derived cell expresses a cell surface marker selected from the group consisting of DC-SIGN, Langerin, CD80, CD86, CD70, CD40, and any combination thereof. In certain embodiments, the allogeneic leukemia-derived cell is CD34-positive, CD1a-positive, CD83-positive, CD80-positive, CD86-positive, and CD40-positive. In some other embodiments, the allogeneic leukemia-derived cell may be CD14-negative. In certain exemplary embodiments, the allogeneic leukemia-derived cell is derived from the DCOne cell line.

In various exemplary embodiments, the subject receives one or more biweekly doses of about 25e6 or about 50e6 allogeneic leukemia-derived dendritic cells. In certain exemplary embodiments, the subject receives one or more booster doses, each dose comprising about 10e6 allogeneic leukemia-derived dendritic cells. In certain exemplary embodiments, each booster dose is administered to the subject about 28 days after a previous dose.

In various exemplary embodiments, the subject achieves MRD conversion or disappearance after receiving treatment with the composition comprising the allogeneic leukemia-derived dendritic cells.

In various exemplary embodiments, the composition is administered to the subject by intradermal injection.

In various exemplary embodiments, the subject had previously been treated with by other therapy such as radiation therapy, chemotherapy, etc. In certain exemplary embodiments, the subject is in complete remission (CR).

In another aspect, provided here is a method of treating cancer in a subject in in need thereof, comprising: administering to a subject having measurable residual disease (MRD) one or more initial doses of an allogeneic leukemia-derived dendritic cell vaccine; and administering to the subject one or more booster doses of the allogeneic leukemia-derived dendritic cell vaccine, if an elevated level of a biomarker is achieved in the subject subsequent to the one or more initial doses of the allogeneic leukemia-derived dendritic cell vaccine relative to the biomarker level in the subject prior to the one or more initial doses, wherein the biomarker comprises is chosen from dendritic cells, CD8+CD45RA+ cells, B cells, NK cells, CD4 CD161+ T cells, CD14+CD16− non-inflammatory monocytes, or a combination thereof.

In certain exemplary embodiments, the dendritic cells are chosen from cDC1 and/or cDC2. In certain exemplary embodiments, the dendritic cells comprise CD141+/CLEC9A⁺ cDC1 dendritic cells and/or CD163⁺ cDC2 dendritic cells. In certain exemplary embodiments, the dendritic cells comprise CD141+/CLEC9A⁺ cDC1 dendritic cells and/or CD163+ cDC2 dendritic cells. In some embodiments, the NK cells may be CD56++NK cells and/or CD56+NK cells.

In some embodiments, the cancer is a liquid cancer. In some further embodiments, the liquid cancer is selected from the group consisting of leukemia, lymphoma, myelodysplastic syndrome (MDS) and myeloma. In certain embodiments, the liquid cancer is acute myeloid leukemia (AML).

In various exemplary embodiments the allogeneic leukemia-derived cell comprises WT-1, MUC-1, PRAME, RHAMM, P53, and Survivin. In various exemplary embodiments, the allogeneic leukemia-derived cell comprises a dendritic cell phenotype. In some embodiments, the allogeneic leukemia-derived cell comprises a mature dendritic cell phenotype. In certain exemplary embodiments, the allogeneic leukemia-derived cell comprises a genetic aberration between chromosome 11p15.5 to 11p12. In certain exemplary embodiments, the genetic aberration encompasses about 16 Mb of genomic regions.

In various exemplary embodiments, the allogeneic leukemia-derived cell is CD34-positive, CD1a-positive, and CD83-positive. In certain exemplary embodiments, the allogeneic leukemia-derived cell expresses a cell surface marker selected from the group consisting of DC-SIGN, Langerin, CD80, CD86, CD70, CD40, and any combination thereof. In certain embodiments, the allogeneic leukemia-derived cell is CD34-positive, CD1a-positive, CD83-positive, CD80-positive, CD86-positive, and CD40-positive. In some other embodiments, the allogeneic leukemia-derived cell may be CD14-negative. In certain exemplary embodiments, the allogeneic leukemia-derived cell is derived from the DCOne cell line.

In various exemplary embodiments, the subject receives one or more biweekly doses of about 25e6 or about 50e6 allogeneic leukemia-derived dendritic cells. In certain exemplary embodiments, the subject receives one or more booster doses, each dose comprising about 10e6 allogeneic leukemia-derived dendritic cells. In certain exemplary embodiments, each booster dose is administered to the subject about 28 days after a previous dose.

In various exemplary embodiments, the subject achieves MRD conversion or disappearance after receiving treatment with the composition comprising the allogeneic leukemia-derived dendritic cells.

In various exemplary embodiments, the composition is administered to the subject by intradermal injection.

In various exemplary embodiments, the subject had previously been treated by other therapy such as radiation therapy, chemotherapy, etc. In certain exemplary embodiments, the subject is in complete remission (CR).

In another aspect, provided here is a method of identifying a subject with cancer in remission having measurable residual disease (MRD) who is likely to be responsive to a treatment with an immunogenetic composition, predicting a risk of developing cancer relapse or recurrence, and/or screening a candidate to receive the treatment with the immunogenetic composition, the method comprising: assessing a baseline level of a biomarker in a peripheral blood of the subject before the subject is treated with the immunogenetic composition in comparison with a predetermined reference level of the biomarker; and identifying the subject as being likely to be responsive to the treatment with the immunogenetic composition if the baseline level of the biomarker in the peripheral blood of the subject is greater than the predetermined reference level of the biomarker; wherein the immunogenetic composition comprises allogeneic leukemia-derived dendritic cells; and wherein the biomarker comprises at least one subset of circulating dendritic cells.

In various exemplary embodiments, the baseline level of the biomarker corresponds to the number of the at least one subset of dendritic cells in the peripheral blood of the subject and is assessed by measuring the number of the at least one subset of dendritic cells in a blood sample obtained from the subject before the subject is treated with the immunogenetic composition.

In certain exemplary embodiments, the predetermined level of the biomarker corresponds to a mean number of the at least one subset of dendritic cells in the peripheral blood of one or more separate subjects with cancer in remission having MRD who are not responsive to the treatment with the immunogenetic composition and display cancer relapse after a first cycle of treatment using the immunogenetic composition.

In certain exemplary embodiment, the method further comprising administering to the identified subject with at least one dose of the immunogenetic composition comprising an effective amount of allogeneic leukemia-derived dendritic cells. In certain exemplary embodiments, the immunogenetic composition is administered to the subject according to a regimen comprising: 4 biweekly prime doses of the immunogenetic composition; and optional one booster dose of the immunogenetic composition applied at week 14 or two booster doses of the immunogenetic composition applied at week 14 and week 18; wherein each biweekly prime dose comprises 25E6 or 50E6 allogeneic leukemia-derived dendritic cells and each booster dose comprises 10E6 allogeneic leukemia-derived dendritic cells.

In another aspect, provided here is a method of identifying a subject with cancer in remission having measurable residual disease (MRD) who is likely to be responsive to a treatment with an immunogenetic composition, predicting a risk of developing cancer relapse or recurrence, and/or identifying a need of a continuation treatment with the immunogenetic composition to prevent or delay relapse or recurrence of cancer or reduce the risk of developing cancer relapse, the method comprising: assessing a baseline level of a biomarker in peripheral blood of the subject before treating the subject with the immunogenetic composition; assessing one or more post-treatment levels of the biomarker in peripheral blood of the subject after the subject is treated with the immunogenetic composition according to a first dosage regimen; and identifying the subject as being likely to be responsive to the treatment with the immunogenetic composition and as in need of continuous treatment with the immunogenetic composition to prevent or delay relapse or recurrence of cancer if the one or more post-treatment levels of the biomarker are greater than the baseline level of the biomarker; wherein the immunogenetic composition comprises allogeneic leukemia-derived dendritic cells; and wherein the biomarker comprises at least one subset of circulating dendritic cells.

In certain embodiments, the baseline level of the biomarker corresponds to a mean number of the at least one subset of dendritic cells in the peripheral blood of the subject and is assessed by measuring the number of the at least one subset of dendritic cells in at least one blood samples periodically obtained from the subject before the subject is administered with the immunogenetic composition. In some embodiments, the baseline level of the biomarker corresponds to a mean number of the at least one subset of dendritic cells in the peripheral blood of the subject and is assessed by measuring the number of the at least one subset of dendritic cells in at least one blood samples periodically obtained from the subject before the subject is administered with the immunogenetic composition, wherein each of the one or more post-treatment levels of the biomarker corresponds to a mean or average number of the at least one subset of dendritic cells in the peripheral blood of the subject and is assessed by measuring the number of the at least one subset of dendritic cells in a blood sample obtained from the subject after the subject is treated with treated with the immunogenetic composition according to the first dosage regimen.

In certain exemplary embodiments, the one or more post-treatment levels of the biomarker include a first post-treatment level and a second post-treatment level of the biomarker in the peripheral blood of the subject, the first post-treatment level of the biomarker is assessed two days after completion of the first dosage regimen, and the second post-treatment level of the biomarker is assessed two weeks after completion of the first dosage regimen.

In certain exemplary embodiments, the method further comprises identifying the subject as in need of a continuous treatment with the immunogenetic composition for at least one booster regimen if the second post-treatment level of the biomarker is greater than the first post-treatment level of the biomarker.

In another aspect, disclosed herein is a method of identifying a subject with cancer in remission having measurable residual disease (MRD) as being likely to be responsive to a treatment with an immunogenetic composition and treating the identified subject to prevent or delay relapse or recurrence of cancer, and/or reduce a risk of relapse or recurrence of cancer, the method comprising: assessing a baseline level of a biomarker in a peripheral blood of a subject before the subject is treated with the immunogenetic composition in comparison with a predetermined reference level of the biomarker; identifying the subject as being likely to be responsive to the treatment with the immunogenetic composition if the level of baseline level of the biomarker in the peripheral blood of the subject is greater than the predetermined reference level of the biomarker; and administering to the identified subject with at least one dose of the immunogenetic composition comprising an effective amount of allogeneic leukemia-derived dendritic cells; wherein the biomarker is chosen from dendritic cells, CD8+ T cells, B cells, NK cells, CD4 CD161+ T cells, CD14+CD16− non-inflammatory monocytes, or a combination thereof.

In certain embodiments, the predetermined level of the biomarker corresponds to a mean number of the at least one subset of dendritic cells in the peripheral blood of one or more separate subjects with the cancer in remission having MRD who are not responsive to the treatment with the immunogenetic composition and display cancer relapse after a first cycle of treatment using the immunogenetic composition.

In another aspect, disclosed herein is a method of identifying a subject with cancer in remission having measurable residual disease (MRD) who has been treated with an immunogenetic composition according to a first dosage regimen and is likely in need of continuous or booster treatment with the immunogenetic composition, and treating the subject to prevent or delay relapse or recurrence of cancer, and/or reduce a risk of relapse or recurrence of cancer, the method comprising: assessing a baseline level of a biomarker in peripheral blood of the subject before the subject is treated with the immunogenetic composition; assessing one or more post-treatment levels of the biomarker in peripheral blood of the subject after the subject is treated with the immunogenetic composition according to a first dosage regimen; identifying the subject as being likely to remain in CR with MRD or have an MRD response to the treatment with the immunogenetic composition and as in need of continuous treatment with the immunogenetic composition to prevent or delay relapse or recurrence of cancer if the one or more post-treatment levels of the biomarker are greater than the baseline level of the biomarker level; and administering to the identified subject with the immunogenetic composition according to a second booster regimen; wherein the immunogenetic composition comprises allogeneic leukemia-derived dendritic cells; and wherein the biomarker is chosen from dendritic cells, CD8+ T cells, B cells, NK cells, CD4 CD161+ T cells, CD14+CD16− non-inflammatory monocytes, or a combination thereof.

In certain exemplary embodiments, the baseline level of the biomarker corresponds to a mean number of the at least one subset of dendritic cells in the peripheral blood of the subject and is assessed by measuring the number of the at least one subset of dendritic cells in at least one blood samples periodically obtained from the subject before the subject is administered with the immunogenetic composition; and the one or more post-treatment levels of the biomarker corresponds to a mean or average number of the at least one subset of dendritic cells in the peripheral blood of the subject and is assessed by measuring the number of the at least one subset of dendritic cells in a blood sample obtained from the subject after the subject is treated with the immunogenetic composition according to the first dosage regimen.

In some exemplary embodiment, the one or more post-treatment levels of the biomarker include a first post-treatment level and a second post-treatment level of the biomarker in the peripheral blood of the subject; wherein the first post-treatment level of the biomarker is assessed two days after completion of the first dosage regimen; and wherein the second post-treatment level of the biomarker is assessed two weeks after completion of the first dosage regimen.

In certain embodiments, the method further comprises identifying the subject as in need of a continuous treatment with the immunogenetic composition for at least one booster regimen if the second post-treatment level of the biomarker is greater than the first post-treatment level of the biomarker.

In certain embodiments, the method the first dosage regimen includes 4 biweekly doses of the immunogenetic composition and two booster doses of the immunogenetic composition applied at week 14 and week 18, wherein each biweekly dose comprises 25E6 or 50E6 allogeneic leukemia-derived dendritic cells, and wherein each booster dose comprises 10E6 allogeneic leukemia-derived dendritic cells. In certain embodiments, the second booster regimen comprises at least one booster dose of the immunogenetic composition applied after week 18. In certain embodiments, each booster dose comprises 10E6 allogeneic leukemia-derived dendritic cells. In certain exemplary embodiments, the method of any one of claims 62-67, wherein each booster dose in the second booster regimen is applied to the subject once every 4 weeks.

In certain exemplary embodiments, the subject is treated with the immunogenetic composition for at least one booster dose until the subject shows MRD conversion or disappearance.

In certain exemplary embodiments, the immunogenetic composition is applied to the subject via an intradermal route. In certain embodiments, the immunogenetic composition is applied to the subject via injection. In some embodiments, the immunogenetic composition is applied to the subject via intradermal rejection.

In certain exemplary embodiment, the biomarker used in the above various embodiments is chosen from dendritic cells, CD8+CD45RA+ cells, B cells, NK cells, CD4 CD161+ T cells, CD14+CD16− non-inflammatory monocytes, or a combination thereof. In certain exemplary embodiments, the dendritic cells may be conventional dendritic cells DC1 (cDC1), conventional DC2 (cDC2), plasmacytoid dendritic cells (pDC), or a combination thereof. In some embodiments, the dendritic cells may be CD141+/CLEC9A+ cDC1, cDC2, CD163+ cDC2, HLA-DR+CD123+ pDC cells, or combination thereof. In some embodiments, the NK cells may be CD56++NK cells and/or CD56+NK cells.

In some embodiments, the cancer in the above various embodiments is a liquid cancer. In some further embodiments, the liquid cancer is selected from the group consisting of leukemia, lymphoma, myelodysplastic syndrome (MDS), myeloma, and a combination thereof. In certain embodiments, the liquid cancer is acute myeloid leukemia (AML).

In some embodiments, the immunogenetic composition used in the above embodiments further comprises a pharmaceutically acceptable carrier, adjuvants, excipients, and/or diluents.

In various exemplary embodiments, the allogeneic leukemia-derived cell used in the above various embodiments is CD34-positive, CD1a-positive, and CD83-positive. In certain exemplary embodiments, the allogeneic leukemia-derived cell expresses a cell surface marker selected from the group consisting of DC-SIGN, Langerin, CD80, CD86, CD70, CD40, and any combination thereof. In certain embodiments, the allogeneic leukemia-derived cell is CD34-positive, CD1a-positive, CD83-positive, CD80-positive, CD86-positive, and CD40-positive. In some other embodiments, the allogeneic leukemia-derived cell may be CD14-negative. In certain exemplary embodiments, the allogeneic leukemia-derived cell is derived from the DCOne cell line.

In various exemplary embodiments, the allogeneic leukemia-derived cell used in the above various embodiments comprise a tumor associated antigen or a nucleic acid encoding a tumor associated antigen that is associated with a cancer cell in the subject. In certain embodiments, the tumor associated antigen is chosen from WT-1, MUC-1, RHAMM, PRAME, p53, Survivin, or combinations thereof. In certain embodiments, the allogeneic leukemia-derived cells comprise at least one tumor associated antigen or a nucleic acid encoding at least one tumor associated antigen that is not associated with a cancer cell in the subject. In certain embodiments, the allogeneic leukemia-derived cells have been inactivated, optionally, via irradiation.

In various exemplary embodiments, the subject in the above various embodiments receives one or more biweekly doses of about 25e6 or about 50e6 allogeneic leukemia-derived dendritic cells. In certain exemplary embodiments, the subject receives one or more booster doses, each dose comprising about 10e6 allogeneic leukemia-derived dendritic cells. In certain exemplary embodiments, each booster dose is administered to the subject about 28 days after a previous dose.

In various exemplary embodiments, the subject achieves MRD conversion or disappearance after receiving treatment with the composition comprising the allogeneic leukemia-derived dendritic cells.

In certain embodiments, the composition used in the above various embodiment is administered to the subject by intradermal injection. In various exemplary embodiments, the subject may have previously been treated with allogeneic leukemia-derived dendritic cells. In certain exemplary embodiments, the subject is in complete remission (CR).

In the above various exemplary embodiments, the liquid cancer may be chosen from chronic myeloid leukemia, acute myeloid leukemia, acute myeloid leukemia, myelodysplastic syndrome (MDS), and myeloma, or a combination thereof. In some embodiments, the cancer may be acute myeloid leukemia (AML). In certain situations, the subject is ineligible for accepting hematopoietic stem cell transplantation. In certain situations, the subject has been initially treated with a chemotherapy and/or debulking surgery, and the remission is induced by the chemotherapy and/or the debulking surgery.

In the above various exemplary embodiments, the subject may be a non-human animal. In certain exemplary embodiments, the subject is a human.

In various embodiments, a subject that would benefit from a method disclosed may have cancer with MRD. The subject may be in a complete or partial recession condition after an initial treatment for cancer. In certain embodiment, the initial treatment may include surgery, chemotherapy, and/or radiation therapy. In certain embodiments, the initial treatment comprises debulking surgery. In certain embodiments, the initial treatment comprises chemotherapy and debulking surgery.

In some embodiments, a method provided herein comprises administering to a subject one or more doses of an effective amount of an immunogenic composition comprising an allogeneic leukemia-derived cell. In some embodiments, each dose of an immunogenic composition comprises from about 10 million to about 25 million allogeneic leukemia-derived cells (e.g., allogeneic leukemia-derived cells as described herein). For example, each dose of an immunogenic composition comprises about 1 million, about 2 million, about 3 million, about 4 million, about 5 million, about 6 million, about 7 million, about 8 million, about 9 million, about 10 million, about 11 million, about 12 million, about 13 million, about 14 million, about 15 million, about 16 million, about 17 million, about 18 million, about 19 million, about 20 million, about 21 million, about 22 million, about 23 million, about 24 million, about 25 million, about 26 million, about 27 million, about 28 million, about 29 million, about 30 million, about 31 million, about 32 million, about 33 million, about 34 million, about 35 million allogeneic leukemia-derived cells. In some embodiments, each dose of the immunogenic composition comprises from about 1 million to about 35 million allogeneic leukemia-derived cells, or any interval therebetween. In certain embodiments, each dose of the immunogenic composition comprises about 10 million allogeneic leukemia-derived cells. In certain embodiments, each dose of an immunogenic composition comprises about 25 million allogeneic leukemia-derived cells.

In some embodiments, one or more doses of an immunogenic composition comprising an allogeneic leukemia-derived cell is administered to the subject. For example, one dose, two doses, three doses, four doses, five doses, six doses, seven doses, eight doses, nine doses, ten doses, eleven doses, twelve doses, or more of the immunogenic composition comprising an allogeneic leukemia-derived cell is administered to the subject. Each of the one or more doses may contain substantially the same number of allogeneic leukemia-derived cells, or may contain different numbers of allogeneic leukemia-derived cells. In certain embodiments, a method for treating a liquid cancer provided herein comprises administering to a subject at least one dose of an effective amount of an immunogenic composition comprising an allogeneic leukemia-derived cell. In certain embodiments, a method for treating a liquid cancer provided herein comprises administering to the subject four doses of the immunogenic composition, wherein each of the four doses comprises about 25 million allogeneic leukemia-derived cells. In certain embodiments, a method for treating a liquid cancer provided herein further comprises administering to the subject two doses of the immunogenic composition, wherein each of the two doses comprise about 10 million allogeneic leukemia-derived cells. As such, in certain embodiments, a subject receives at least six doses of the immunogenic composition, four doses each having about 25 million allogeneic leukemia-derived cells, and two doses each having about 10 million allogeneic leukemia-derived cells. Accordingly, in certain embodiments a subject is administered a total of about 120 million allogeneic leukemia-derived cells. In some embodiments, a subject is administered a total of from about 50 million to about 200 million allogeneic leukemia-derived cells, e.g., about 50 million cells, about 60 million cells, about 70 million cells, 80 million cells, about 90 million cells, about 100 million cells, about 110 million cells, about 120 million cells, about 130 million cells, about 140 million cells, about 150 million cells, about 160 million cells, about 170 million cells, about 180 million cells, about 190 million cells, about 200 million cells, or any number of cells therebetween.

In some embodiments, doses of the immunogenic compositions (i.e., comprising an allogeneic leukemia-derived cell) may be administered at an interval of time, e.g., at 1 week intervals, at 2 week intervals, at 3 week intervals, at 4 week intervals, at 5 week intervals, at 6 week intervals, at 7 week intervals, at 8 week intervals, at 9 week intervals, at 10 week intervals, at 11 week intervals, at 12 week intervals, or longer. In some embodiments, the time between doses is from about 1 day to about 21 days, from about 1 day to about 22 days, from about 1 day to about 23 days, from about 1 day to about 24 days, from about 1 day to about 3 weeks, from about 1 day to about 4 weeks, from about 1 day to about 5 weeks, from about 1 day to about 10 weeks, from about 1 day to about 15 weeks, from about 1 day to about 20 weeks, from about 1 day to about 25 weeks, from about 1 day to about 30 weeks, from about 1 day to about 35 weeks, from about 1 day to about 40 weeks, from about 1 day to about 45 weeks, from about 1 day to about 50 weeks, from about 1 day to about 1 year, and any intervening amount of time thereof. In some embodiments, the time between doses is about 1 day to about 1 month, 14 days to about 2 months, 1 month to about 3 months, 2 months to about 5 months, 4 months to about 6 months, 5 months to about 7 months, 6 months to about 8 months, 7 months to about 9 months, 8 months to about 10 months, 9 months to about 11 months, 10 months to about 12 months, 11 months to about 13 months, 12 months to about 14 months, 13 months to about 15 months, 14 months to about 16 months, 15 months to about 17 months, 16 months to about 18 months, 17 months to about 19 months, 18 months to about 20 months, 19 months to about 21 months, 20 months to about 22 months, 21 months to about 23 months, 22 months to about 24 months, 3 months to about 1 year, 6 months to about 1 year, and any intervening range of time thereof.

The methods provided herein are suitable for treating a liquid cancer, e.g., leukemia, lymphoma, myelodysplastic syndrome (MDS), and/or myeloma. In certain embodiments, a method for treating a liquid cancer provided herein is useful for treating acute myeloid leukemia.

Methods described herein comprises administering to the subject an effective amount of an immunogenic composition comprising an allogeneic leukemia-derived cell. As described above, methods described herein include methods comprising the administration of one or more doses of the immunogenic composition. In some embodiments, the one or more doses are administered via the same route of delivery. In some embodiments, the one or more doses are administered via different routes of delivery.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific embodiments thereof, it should be understood by those skilled in the art that various changes may be made, and equivalents may be substituted without departing from the true spirit and scope of the disclosure. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### D. PHARMACEUTICAL COMPOSITIONS AND FORMULATIONS

Also provided are immunogenic compositions comprising an allogeneic leukemia-derived cell of the present disclosure, including pharmaceutical compositions and formulations, such as unit dose form compositions. The pharmaceutical compositions and formulations generally include one or more optional pharmaceutically acceptable carrier or excipient. In certain embodiments, the composition includes at least one additional therapeutic agent (e.g., a second therapy having cytostatic or anticancer activity). Therapies of the present disclosure can be constituted in a composition, e.g., a pharmaceutical composition (e.g., an immunogenic pharmaceutical composition) containing an allogeneic leukemia-derived cell and optionally a pharmaceutically acceptable carrier.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. Accordingly, there are a variety of suitable formulations. A pharmaceutically acceptable carrier includes, but is not limited to, a buffer, excipient, stabilizer, or preservative. In certain embodiments, the choice of carrier is determined in part by the particular cell and/or by the method of administration. A pharmaceutically acceptable carrier includes any and all solvents, dispersion media, coatings, antibacterial and antifungal agents, isotonic and absorption delaying agents, and the like that are physiologically compatible. In certain embodiments, the carrier for a composition containing an allogeneic leukemia-derived cell is suitable for intravenous, intramuscular, subcutaneous, parenteral, spinal or epidermal administration (e.g., by injection or infusion). In certain embodiments, where suitable, e.g., a small molecule based second therapy, the carrier for a composition containing the second therapy is suitable for non-parenteral, e.g., oral administration. A pharmaceutical composition of the disclosure can include one or more pharmaceutically acceptable salts, antioxidant, aqueous and non-aqueous carriers, and/or adjuvants such as preservatives, wetting agents, emulsifying agents and dispersing agents. In some embodiments, the pharmaceutical composition can contain preservatives. Suitable preservatives may include, for example, methylparaben, propylparaben, sodium benzoate, and benzalkonium chloride. In certain embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in certain embodiments are included in the compositions. Suitable buffering agents include, for example, citric acid, sodium citrate, phosphoric acid, potassium phosphate, and various other acids and salts. In certain embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, e.g., those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In certain embodiments, the cell populations are administered parenterally. The term “parenteral,” as used herein, includes intravenous, intramuscular, subcutaneous, rectal, vaginal, and intraperitoneal administration. In certain embodiments, the cells are administered to the subject using peripheral systemic delivery by intravenous, intraperitoneal, or subcutaneous injection.

In certain embodiments, a method for treating a liquid cancer comprises administering an immunogenic composition comprising an allogeneic leukemia-derived cell, wherein the immunogenic composition further comprises a pharmaceutically acceptable carrier. In certain embodiments, the immunogenic composition is formulated for intradermal administration. In certain embodiments, the administration of the immunogenic composition is intradermal.

Compositions in certain embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations. The formulations to be used for in vivo administration are generally sterile. Sterility may be readily accomplished, e.g., by filtration through sterile filtration membranes.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about using agents delaying absorption, for example, aluminum monostearate and gelatin.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific embodiments thereof, it should be understood by those skilled in the art that various changes may be made and equivalents may be substituted without departing from the true spirit and scope of the disclosure. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto.

The embodiments of invention disclosed herein are illustrated herein by the experiments described by the following non-limiting examples. It should be understood that these Examples are given by way of illustration only. From the above discussion and these Examples, one skilled in the art can ascertain the essential characteristics of embodiments of the present invention. Without departing from the spirit and scope thereof, one skilled in the art can make various changes and modifications of the invention to adapt it to various usages and conditions. All publications, including patents and non-patent literature, referred to in this specification are expressly incorporated by reference herein.

### E. EXPERIMENTAL EXAMPLES

As disclosed herein and in all of the following examples:
“MRD response” is defined as a full conversion to negative MRD (i.e., non-detectable level of MRD) or a decrease in MRD levels of at least 1 log 10, i.e., at least a 10-fold decrease in baseline MRD levels.
“pDC” is defined as a DC cell expressing DR+ and CD123+(HLA-DR+, CD123+, pDC).
“cDC1” is defined as CD45+ HLA-DR+CD14−, CD11b−, CD11c+, CD141+, and CLEC9A+.
“cDC2” is defined as CD45+, HLA-DR+, CD14−, CD11b−, CD1c+, CDCl+
“CDCl63+ cDC2” is defined as HLA-DR+, CD14−, CD11b−, CD1c+, and CD163+.

A “patient who relapsed” means that the patient relapsed before or at week 32 after the first dose of vaccination was administered.

A “patient who was stable” means that the patient did not show relapse and remained in remission with MRD positive or had no change in MRD levels.

A “patient with a MRD response” or an “MRD responder” means that the patient, after receiving vaccination, had full conversion (negative MRD, or undetectable levels of tumor cells) or a decrease in MRD levels of at least 1 log 10 detected by genetic and/or flow cytometric testing.

### Example 1—Evaluation of the Efficacy of Immunotherapv of DCP-001 in AML Patients with MRD

Vaccination to prevent relapse in cancer patients is challenging due to the reduced level of immune competence, frequently due to cancer treatment, which in most cases modulates the immune system, either or not in combination with an aging immune system. Vaccination strategies rely on successful processing and presentation of introduced antigens to initiate an adaptive immune response able to kill (residual) tumor cells. Processing of antigens is optimally done by professional antigen presenting cells, like dendritic cells, macrophages, or monocytes.

In this example, Phase II clinical trial study (ADVANCE-II, Clinicaltrials.gov identifier: NCT03697707) were performed to evaluate the efficacy and safety of using an allogeneic leukemia-derived cell (DCP-001 cells) as a cancer vaccine (vididencel) to treat patients with acute myeloid leukemia and MRD to eradicate or control residual cancer cells, and/or prevent or delay cancer relapse. DCP-001 cells and the method of producing these cells are described in U.S. Pat. No. 11,027,001, which is incorporated herein as reference for its entirety.

### 1.1 Vaccine

Dendritic cells are professional antigen-presenting cells (APCs) and exquisitely suited to induce anti-cancer immune responses. The cancer vaccine used in this clinic trial comprises allogeneic leukemia-derived cells DCP-001, which was developed from an acute myeloid leukemia (AML)-derived cell line that uniquely combines the positive features of allogeneic DC vaccines and expression of multiple tumors associated antigens. It was hypothesized that administration of the DCP-001 vaccine after initial treatment would induce anti-cancer immune responses and advance eradication of residual cancer cells within a fully formed suppressive environment, and thus prevent or delay cancer relapse or recurrence.

The tumor associated antigens (TAA's) presented by DCP-001 were found to be shared across different tumor types. These antigens include, but are not limited to, WT-1, MUC-1, WT1, RHAMM, survivin, p53, and PRAME.

Further, it was observed in the Phase I study that vaccination with DCP-001 in 12 post-remission AML patients having prolonged MRD status was associated with improved progression free survival (PFS) and systemic immunogenicity. (See, van de Loosdrecht et al. Cancer Immunol. Immunother. (2018) 67(10):1505-1518, the disclosure of which is incorporated by reference herein in its entirety.) It was observed that treatment using the vaccine was associated with only limited side-effects such as fever, injection site reactions, adenopathy, and fatigue.

### 1.2 Patients

Twenty adult patients (10 female and 10 male) entered into this Phase II clinical trial. All of these 20 patients were in complete remission (CR1/CRi) after an initial treatment (e.g., standard induction and consolidation therapy (chemotherapy)) and were ineligible for hematopoietic stem-cell transplantation (HSCT). 19 patients were MRD positive at baseline. MRD was assessed on bone marrow aspirates by multicolor flow cytometry (MFC) at a value of >0.1%, and/or by molecular assay for the detection of specific molecular abnormalities such as NPM1 mutation according to European LeukemiaNet (ELN) guidelines.

### 1.3 Vaccination Strategies/Administration Schedule

Two different vaccination regimens of immunotherapy with DCP-001 were tested. The 20 patients were placed in two groups, i.e., Cohort 1 and Cohort 2, with 10 patients in each group. The patients in Cohort 1 were scheduled to receive a primary vaccination schedule comprising four bi-weekly intradermal (i.d.) vaccinations with 25e6 cells per vaccination at week 0, 2, 4, and 6, followed with a boosting schedule comprising 2 booster vaccinations with 10e6 cells per vaccination at week 14 and 18 (in a monthly interval). The patients in Cohort 2 were scheduled to receive four bi-weekly vaccination with 50e6 cells per vaccination at week 0, 2, 4, and 6, followed with 2 booster vaccinations with 10e6 cells per vaccination at week 14 and 18.

Median time from the start of an initial chemotherapy induction for AML to the first dose of DCP-001 was 294 days.

### 1.4 Sample Collection and Methods

### 1) Sample Collection

Patients were evaluated and monitored before, during, and after the vaccination. All patients were followed up to week 70 and long-term follow up is ongoing to 5 years. Peripheral blood samples were collected by vena puncture from each patient at baseline (before receiving the vaccination), and at day 3, 42, 44, week 11, 14, 18, 20, and 32, after the first dose was given. Sera and cell samples from collected blood samples were used for assessment of efficacy (MRD evaluation) and immune response monitoring. Skin biopsies of the site of injection were taken on day 3, 44, and week 18 after the first dose was given.

### 2) MRD Assessment

MRD status was assessed on bone marrow aspirates by flow cytometry and/or qPCR according to ELN guidelines. (Heuser M et al., 2021 Update Measurable Residual Disease in Acute Myeloid Leukemia: European LeukemiaNet Working Party Consensus Document Blood 2021 doi 10.1182/blood.2021013626). MRD response is defined by either a 10-fold decrease in the baseline level of MRD or a conversion to MRD negative (non-detectable.

### 3) Immunomonitorinq of Peripheral Blood Mononuclear Cells

PBMC were isolated, frozen and stored in until used. Samples were taken at baseline, 2 days after the 1st and 4th dose, at week 11, 18, 20 and at week 32.

### 4) IFNγ ELISPOT

Samples were analyzed by restimulation with WT1, PRAME or RHAMM peptide pools. Vaccine induced response (VIR) was defined of at least a 2-fold increase of the specific, mock-corrected background response over baseline. Sustained VIR was defined of at least 2 VIR to the same antigen during treatment (up to week 32)

### 5) Flow Cytometry

Multiparameter flow cytometry was performed using a 40-marker panel on frozen PBMC samples. Immune subset analysis of dendritic (live, single, CD45+, Lin-(CD3, 56, 19), CD16−, CD15−) cells was performed and correlated to clinical response, relapse free and overall survival, by high dimensional analysis using Flow SOM.

### 1.5 Outcome Measures

### 1) Primary Endpoint

MRD status was used as a primary clinical outcome parameter to assess the efficacy of the treatment. Blood samples at baseline and at timepoints of week 14, 20, and 32 after the first dose was given were assessed by flow cytometry and or molecular analysis to detect MRD. The effect of immunotherapy of the allogeneic dendritic cells DCP-001 was evaluated based on the change of MRD at timepoints of week 14, 20, and 32 after the first dose was given as compared to the baseline MRD.

### 2) Secondary Endpoint

Safety, including treatment emergent adverse events and serious adverse events, relapse, relapse-free survival, and overall survival (OS) were monitored throughout the study up to 56 weeks after the first dose was given. Immune responses induced by DCP-001 were monitored up to 32 weeks after the first dose was given.

Systemic immune responses induced by DCP-001 were assessed based on IFN-γ ELISpot assay to tumor associated antigens (WT1, RHAMM, and/or PRAME) in peripheral blood samples collected at baseline and at the timepoints of day 3, 44, week 11, week 18, week 20, and week 32 after the first dose was given.

Immunohistochemistry of skin biopsies of the site of injection was assessed in samples obtained at time points of day 3, 44, and week 18 after the first dose was given.

### 1.6 Results

### 1) MRD Responses

All 20 patients have received 4 initial vaccinations, 17 patients received all booster vaccinations and 1 patient received only one booster. Immune monitoring was performed on all samples collected from the patients, until week 32 or end of study. Vaccination with DCP-001 resulted in substantial MRD responses in 7 patients (35%), where five patients converted to MRD negative (MRD conversion) and 2 patients had a 10-fold reduction, i.e., at least 1 Log 10, in MRD levels. Of the other patients, 7 (35%) remained stable MRD level and 6 patients relapsed within 32 weeks after vaccination (30%).

### 2) Evaluation of Skin Biopsies

Skin biopsies of the site of injection taken on day 3, 44, and week 18 after the first dose was given, corresponding to 2 days after the 1^{st}, 4^{th} or 6^{th} vaccination, were analyzed by flow cytometry to evaluate the immune profiles. The results showed that all patients had moderate to high levels of infiltrating CD4 and CD8 T-cells, as well as CD68 positive cells (macrophages).

### 3) T-Cell Responses Induced by DCP-001

Vaccine induced T cell responses to WT1, PRAME and/or RHAMM were assessed in human peripheral blood mononuclear cells (PBMCs) of all 20 patients by IFNγ ELISPOT assays. In 17 patients received all booster vaccinations, at least 1 vaccine induced response (VIR) to any of these antigens WT1, RHAMM, and/or PRAME was detected.

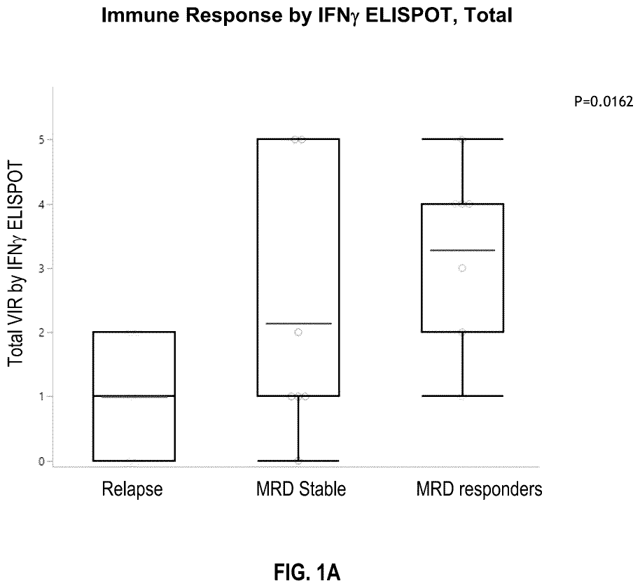

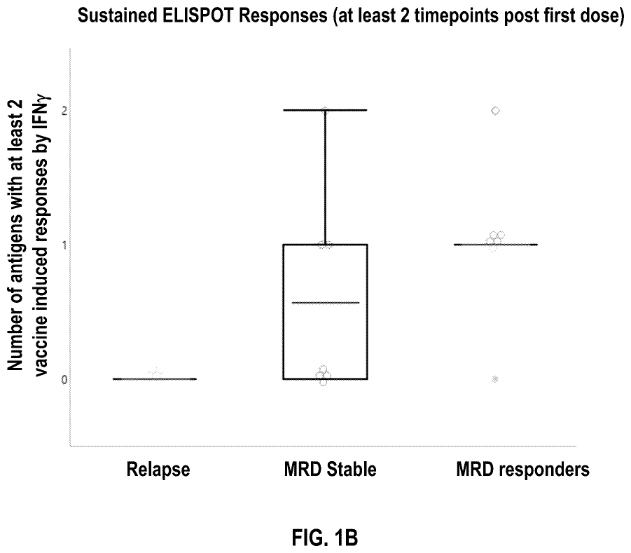

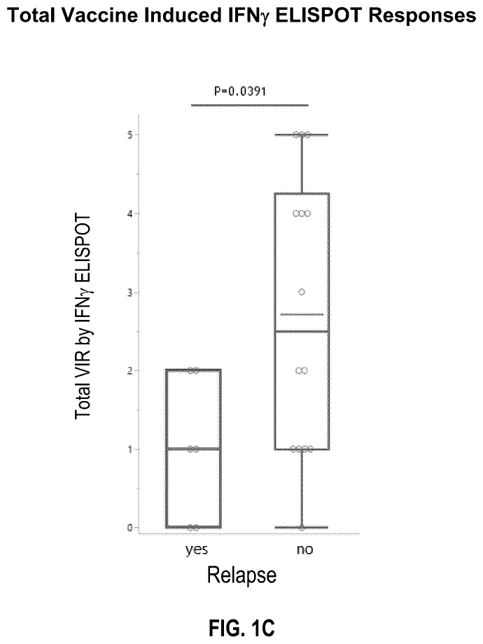

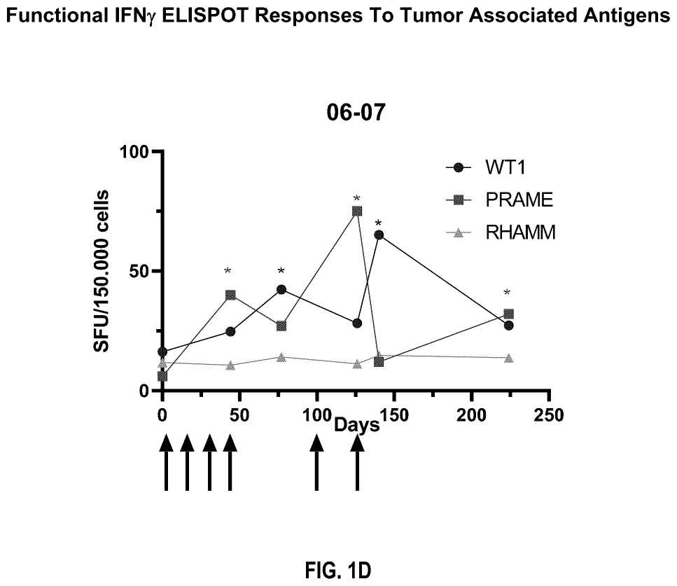

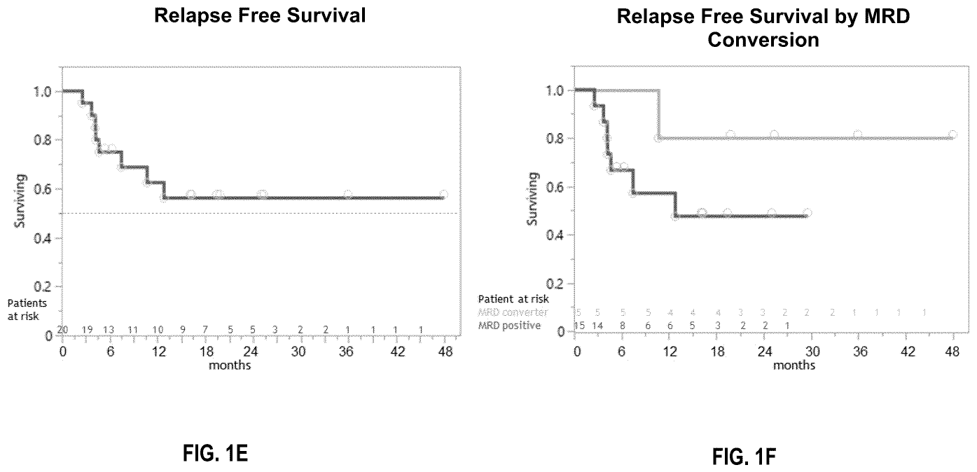

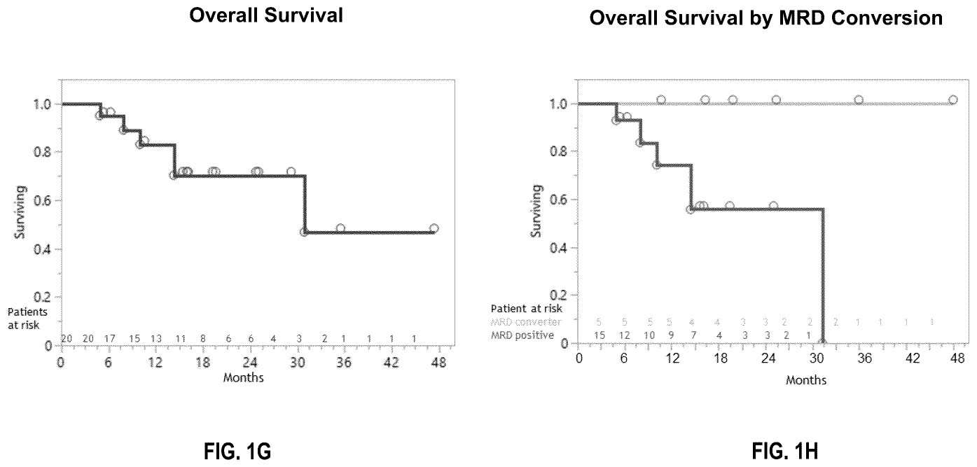

FIG. 1A shows the total immune response induced by the cancer vaccine comprising DCP-001 measured by IFNγ ELISPOT essay. As can be seen in FIG. 1A, ELISPOT response is reflected by the 2-fold increase of the spots over the background corrected baseline response to either of the antigens (WT1, PRAME or RHAMM). MRD responders (MRD converted to be negative) had the highest total number of VIRs, and the total number of VIRs in patients with stable MRD was greater that if patients had relapse.

Durable VIR were counted at least 2 VIR to the same antigen at 2 timepoints after baseline were observed, and the results are shown in FIG. 1B, which illustrates sustained ELISPOT responses at least 2 timepoints post first dose in patients with relapse, patients with stable MRD, and MRD responders. As shown in FIG. 1B, sustained ELISPOT responses were reflected by vaccine induced ELISPOT response with a 2-fold increase over background corrected baseline responses to the same antigen. The results show that sustained VIR were detected in many patients with stable MRD and MRD responders, where the patients who responded to DCP-001 and had MRD converted to negativity (MRD responders) had a higher level of durable VIRs than patients with remained level of MRD, indicating that the MRD responders had a broad and durable T-cell response.

FIG. 1C illustrates vaccine induced responses to tumor associated antigens (either WT1, Prame or Rhamm) in patients with or without relapse during the first 32 weeks of the study. As can be seen in FIG. 1C, an increased number of vaccine-induced responses is observed without relapse.

FIG. 1D is a graph illustrating ELISPOT responses for either of the three antigens, during vaccination with DCP-001 (indicated vaccination timepoints by arrows). Vaccine induced responses are indicated by *. In FIG. 1D, “SFU” represents spot forming units.

These results demonstrate that DCP-001 induced functional T-cell responses are increased and durable in patients with MRD response and there is a clear correlation between the level of VIRs and the clinical MRD response.

### 4) Relapse Free Survival and Overall Survival

FIG. 1E is a graph illustrating the results of relapse free survival of the patients up to 48 months post the 1^{st} dose of the DCP-001. FIG. 1F illustrates the relapse free survival by MRD conversion up to 48 months post the 1^{st} dose of the DCP-001. As can be seen, the median relapse-free survival (RFS) has not yet been reached, with a median follow-up (FU) period of about 17.7 months for the overall study population. Estimated RFS at 12 months is about 64% (41%-80%) and the estimated median RFS is about 17.5 months (9.3-32.7 months).

FIG. 1G is a graph illustrating the results of overall survival (OS) of the patients up to 48 months post the 1^{st} dose of the DCP-001. FIG. 1H illustrates the overall survival by MRD conversion up to 48 months post the 1^{st} dose of the DCP-001. As can be seen, with a median follow up (FU) period the median FU overall population is about 17.7 months, while the current median OS is about 30.9 months (2.6 years), with estimated 85.3% of OS (65-94%) at 12-months, and estimated 66.3% (40-83%) of OS at 24 months.

To date, median RFS and OS have not yet been reached, with a median follow up period of 15.4 (range 6.6-16.8) months for the entire study population. Estimated RFS and OS at 6 months is 83.9% (range 64-94%) and 97% (range 79-99%), respectively. Estimated RFS and OS at 12 months is 70% (range 48-85%) and 87% (67-95%), respectively.

The above results show that intradermal administration of DCP-001 was well-tolerated and safe. In addition, the results show that administration of DCP-001 led to functional T-cell responses against TAAs (WT-1, PRAME, RHAMM) with more vaccine-induced responses in patients who showed clinical benefit or had an MRD response. Treatment les to MRD conversion (MRD-) or reduction in MRD in 7/20 patients, which was correlated with number of VIRs against relevant TAAs. The results demonstrate that vaccination with DCP-001 is a very promising immune therapy modality for AML maintenance treatment after chemotherapy induction, specifically for MRD+patients.

### Example 2—Identify Biomarkers for Predicting Clinic Response to DCP-001

In this example, the statuses of immune cell compositions in the blood samples from patients that participated in Phase II clinical trial as described in Example 1 were analyzed by flow cytometry. The statuses of the immune cell compositions were compared between patients who showed cancer relapse, patients who did not show relapse but had stable MRD, and patients who had MRD response (converting to MRD negativity), as well as between time points before, during, and/or after the vaccination with DCP-001.

In the following figures of this Example, number “1,” when present and being used to indicate the patients in group, represents patients who relapsed (only for 2 patients' samples are available for analysis at V13, i.e., week 32 after the first dose of vaccination was administered); number “2,” when present and being used to indicate the patients in group, represents patients who did not show relapse but had stable MRD; number “3,” when present and being used to indicate the patients in group, represents patients who had MRD response.

In the following figures of this Example, boxplots, if present, are shown with median and 25% and 75% quantiles and the min/max. The lines in the boxplots represent the mean. The mean is in most cases comparable to the median, indicating that outliers are not present or do not influence to a large extent the data.

Multiparameter flow cytometry was performed using a 40-marker panel on frozen PBMC samples obtained in Example 1. Analysis was performed using Flow Jo for live, CD45+ cells, followed by gating for the different immune cells as B-, CD4, CD8 T-cells and NK cells, myeloid cells, DC subsets, T-cell memory, activation and exhaustion markers.

### 2.1 Baseline Immune Cell Compositions

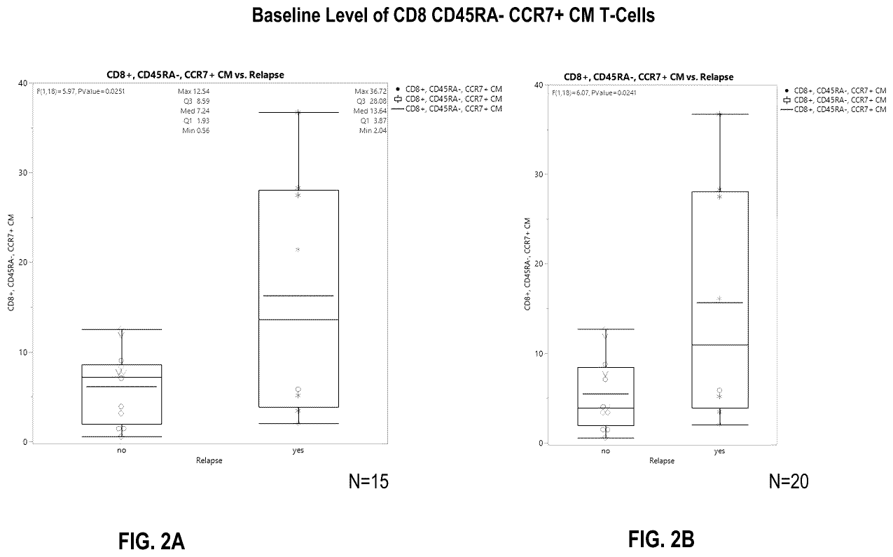

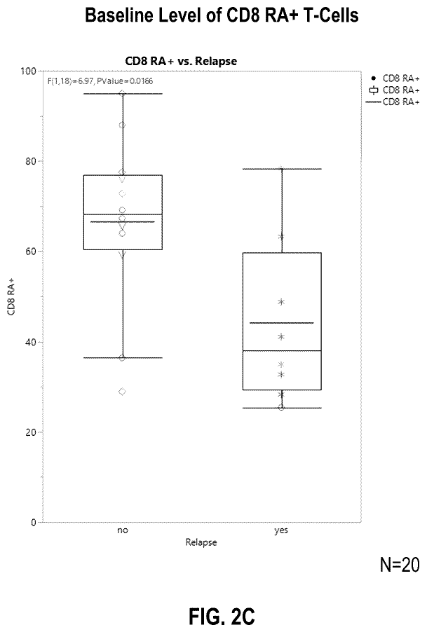

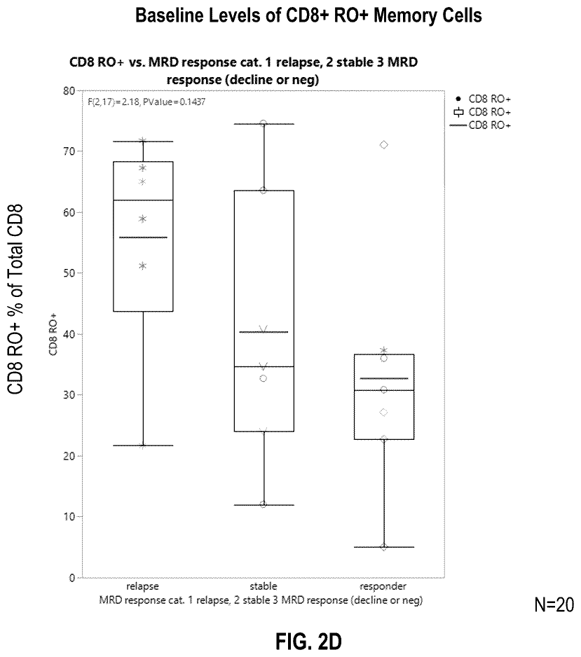

Immune cell compositions in baseline blood samples from patients that participated in the Phase II clinic trial described in Example 1 were assessed by flow cytometry. Baseline statuses of the patients' immune system were evaluated using statistics program JMP@ with a comparison between patients who relapsed and who did not relapse (including patients who remained in complete remission with MRD (CR MRD+) and who converted to MRD negativity (MRD response)). The results are illustrated in FIGS. 2A, 2B, 2C, and 3-6.

### 1) Baseline Level of CD8 T Cells

FIGS. 2A-2D depict graphs illustrating the baseline levels of CD8 T cells of patients who relapsed compared to patients who did not relapse. FIG. 2A and FIG. 2B illustrate the baseline levels of the population of CD8+T memory cells (CM) without leukocyte common antigen isoform CD45RA and with the chemokine receptor CCR7 (CD8+, CD45RA−, CCR7+, CM) in patients who relapsed and patients who did not relapse, measured in 15 patients and 20 patients, respectively. FIG. 2C illustrates the baseline levels of the population of CD8 RA+ T cells in patients who relapsed compared to patients who did not relapse. FIG. 2D illustrates the baseline levels of the population of CD8+RO+ Memory Cells in patients who relapsed, remained in CR with MRD, and MRD response, respectively.

As can be seen in FIGS. 2A-2D, patients who relapsed had a higher baseline level of CD8+, CD45RA−, CCR7+, CM T cells and CD8 RO+ T cells, and lower level of CD8+CD45RA+ T cells, compared to patients who did not relapse.

### 2) Baseline Level of B Cells

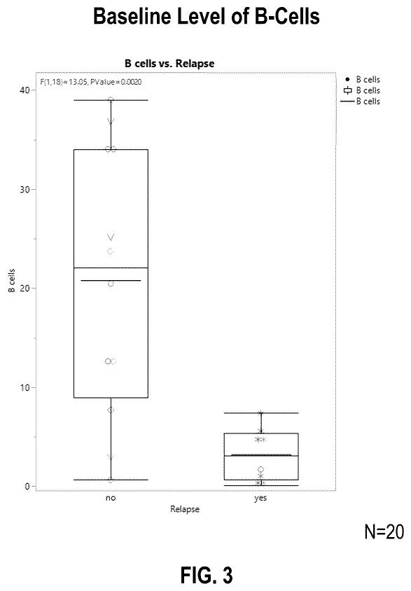

FIG. 3 depicts graphs illustrating the baseline levels of B cells in patients who relapsed compared to patients who did not relapse. As can be seen in FIG. 3, patient who relapsed had a lower number of B cells, compared to patients who did not relapse.

3) Baseline Level of cDC2 Cells

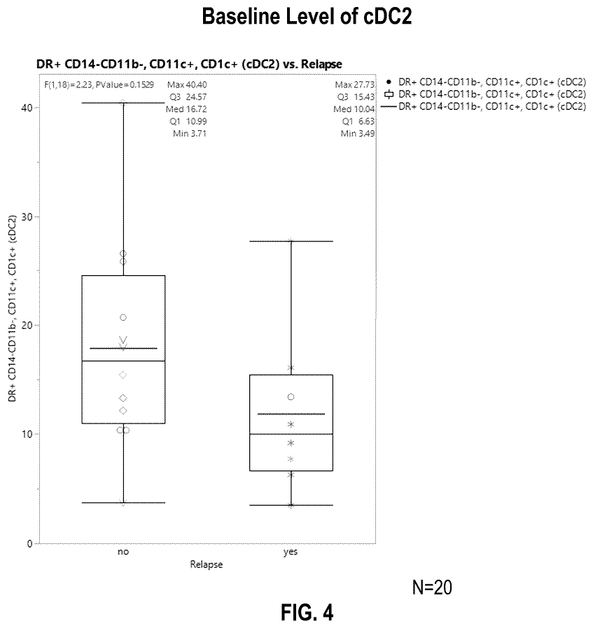

FIG. 4 depicts graphs illustrating the baseline levels of cDC2 (HLA-DR+, CD14−, CD11b−, CD11C+) cells in patients who relapsed compared to patients who did not relapse. As can be seen in FIG. 2, patient who relapsed had a lower number of cDC2 cells than patients who did not relapse.

### 4) Baseline Level of NK Cells

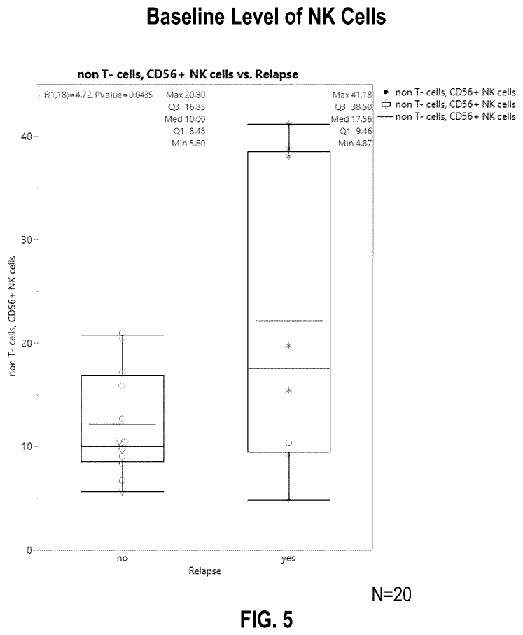

FIG. 5 depicts graphs illustrating the baseline levels of NK cells (non T-cells, CD56+) in patients who relapsed compared to patients who did not relapse. As can be seen in FIG. 5, patient who relapsed had a higher number of NK CD56+ cells than patients who did not relapse.

### 5) Baseline Level of CD4 CD161+ Cells

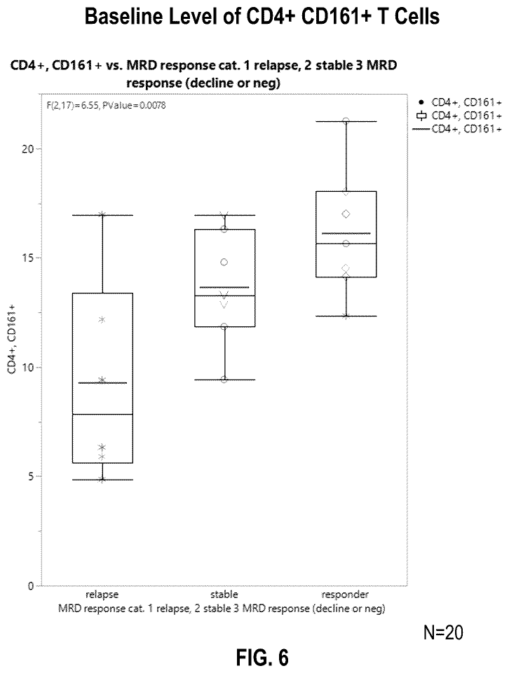

FIG. 6 depicts graphs illustrating the baseline levels of CD4 CD161+ T cells in patients who relapsed, patients who remained in CR with MRD, and patients with MRD response. As can be seen in FIG. 6, patient who relapsed had a lower number of CD4 CD161+ T cells compared to patents who did not show relapse.

The above results show that patients with stable MRD levels or those with a MRD response had a higher number of circulating DC (pDC, cDC1 and cDC2) at baseline compared to patients who relapsed. These results demonstrate that the baseline statuses of the patients' immune system correlated with the clinical response to vaccination with DCP-001.

It has been observed that baseline demographics do not show a marked difference in time from start induction to first dose or time from last consolidation to first DCP-001 dose on the clinical response.

2.2 Immune Cell Compositions in Response to Vaccination with DCP-001

During vaccination with DCP001 and clinical follow-up, immune cell composition in blood samples collected from 15 patients who relapsed and who did not relapse (including patients who remained in CR with MRD and who converted to MRD negativity (MRD response)), were assessed by flow cytometry and evaluated using statistics program JMP in comparison with baseline levels. All of these 15 patients have received full doses of vaccination with DCP-001. It has been found that several immune cell populations were significantly different post-vaccination as compared to baseline status. These immune cell populations create a distinctly immune profile for patients who relapsed versus those who remained in complete remission with stable MRD (CR MRD+) and those who converted to MRD negativity (MRD response).

1) Assessment of Response of Dendritic Cells cDC1 (HLA-DR+, CD14−, CD11b−, CD11c+, and CD141+/CLEC9A+) in Immune Response to Vaccination with DCP-001

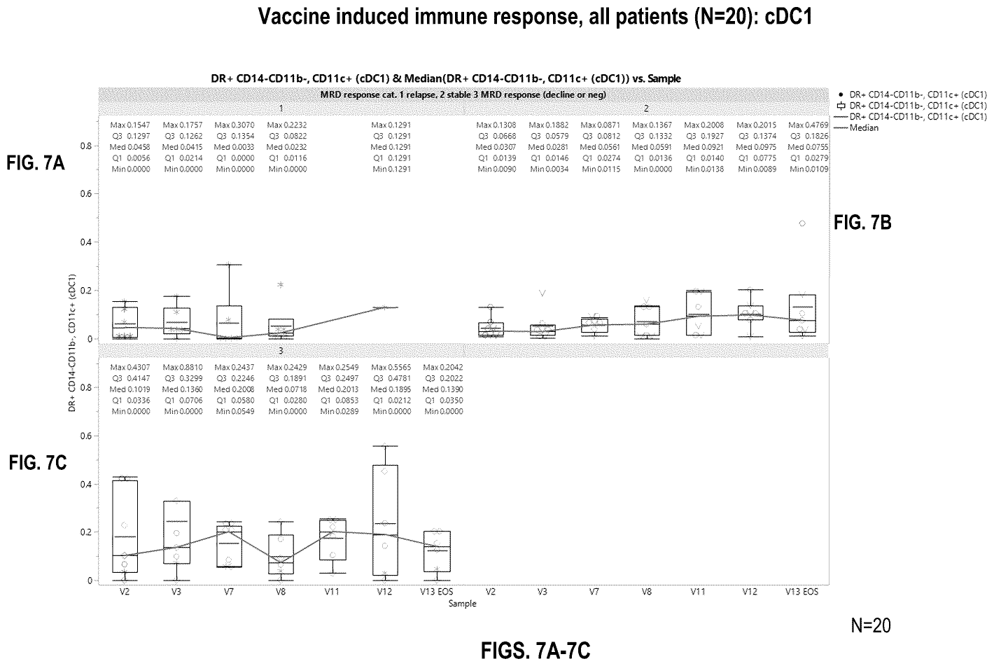

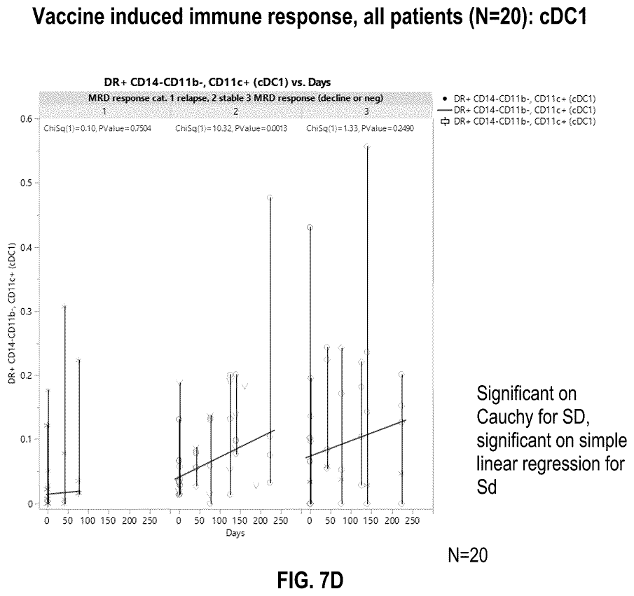

FIGS. 7A-7D depict graphs illustrating the status of dendritic cells cDC1 (HLA-DR+, CD14−, CD11B−, CD11c+) in blood samples collected from the patients before, during, and after the course of vaccination with DCP-001 as described in Example 1, where FIG. 7A illustrates the status of the cDC1 cells in patients who relapsed, FIG. 7B illustrates the status of the cDC1 cells in patients who remained in complete remission with stable MRD, and FIG. 7C illustrates the status of the cDC1 cells in patients who had an MRD response. In FIGS. 7A-7C, V2 corresponds to baseline (day 0), V3 corresponds to day 3, V7 corresponds to day 42, V8 corresponds to day 77 (week 11), V11 corresponds to week 18, V12 corresponds to week 20, and V13 corresponds to week 32. FIG. 7D illustrates significance analysis of the level of cDC1 (HLA-DR+, CD14−, CD11B−, CD11c+) in the blood of the patients before, during, and after the course of the vaccination with DCP-001.

As shown in FIGS. 7A-7C and demonstrated by the simple linear regression for SD in FIG. 7D, the level of the cDC1 cells in patients who remained in complete remission with stable MRD and who had an MRD response increased significantly during and after the course of the vaccination with DCP-001, while the level of cDC1 in patients who had relapse did not show significant change. In addition, the level of the cDC1 cells in patients who relapsed was overall significantly lower than those in patients who remained in complete remission with stable MRD and who had an MRD response.

It has also been observed that patients who had an MRD response had higher levels of CD141+/CLEC9A+ cDC1 cells at baseline, while patients with lower levels of CD141+/CLEC9A+ dendritic cells at baseline had no change of the MRD status or relapsed. Very low levels of CD141+/CLEC9A+ at baseline, resulted in 50% of the cases in very quick relapse, before completion of the vaccination regimen.

2) Assessment of Dendritic Cells cDC2 (HLA-DR+, CD14−, CD11b−, CD11c+) in Immune Response to Vaccination with DCP-001

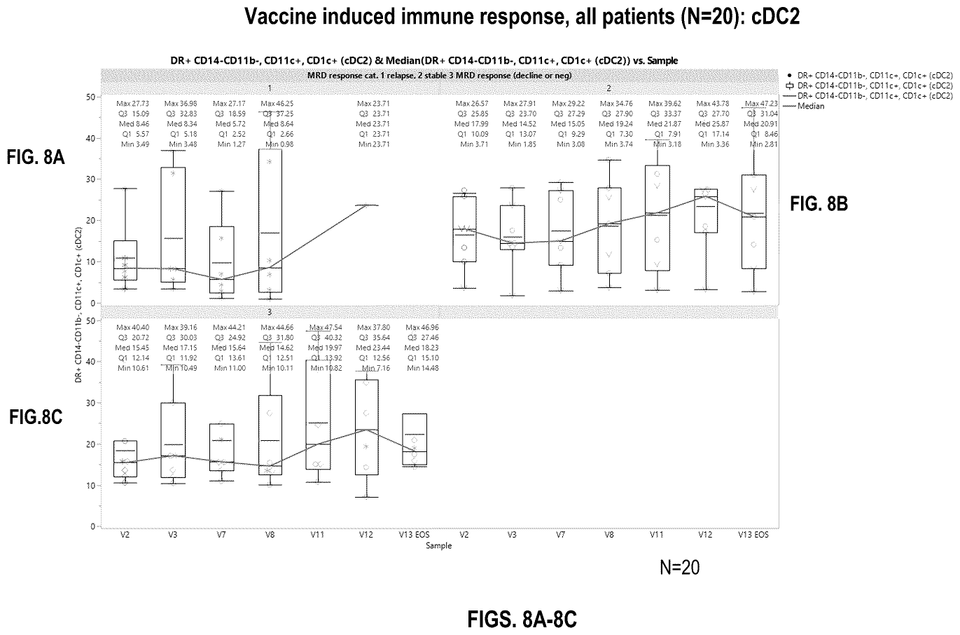

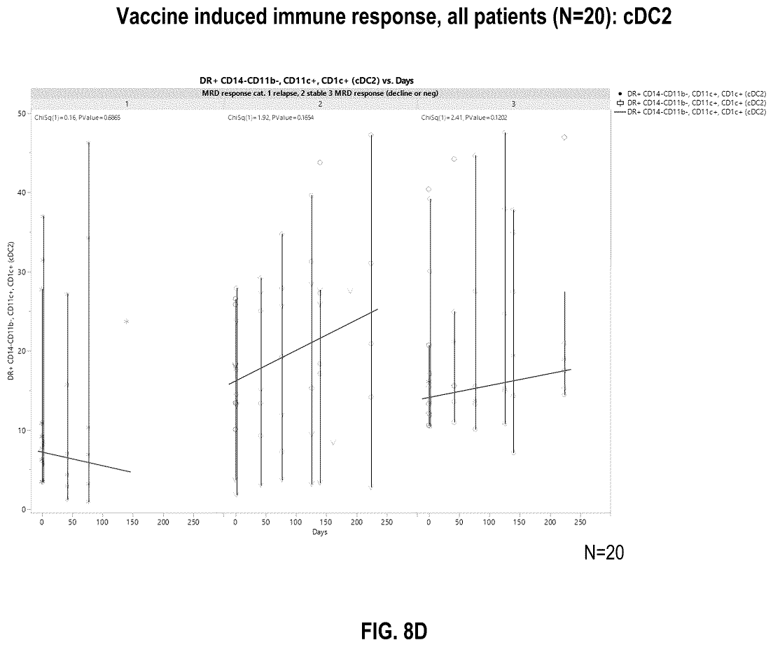

FIGS. 8A-8D depict graphs illustrating the status of dendritic cells cDC2 (HLA-DR+, CD14−, CD11b−, CD11c+) in blood samples collected from the patients before, during, and after the course of the vaccination with DCP-001, as described in Example 1, where FIG. 8A illustrates the status of the cDC2 cells in patients who relapsed, FIG. 8B illustrates the status of the cDC2 cells in patients who remained in complete remission with stable MRD, and FIG. 8C illustrates the status of the cDC2 cells in patients who had an MRD response. In FIGS. 8A-8C, V2 corresponds to baseline (day 0), V3 corresponds to day 3, V7 corresponds to day 42, V8 corresponds to day 77 (week 11), V11 corresponds to week 18, V12 corresponds to week 20, and V13 corresponds to week 32. FIG. 8D illustrates the level of cDC2 (HLA-DR+, CD14−, CD11B−, CD11c+) in the blood of the patients before, during, and after the course of the vaccination with DCP-001.

As shown in FIGS. 8A-8D the level of the cDC2 cells in patients who remained in complete remission with stable MRD and who had an MRD response continuously increased during and after the course of the vaccination with DCP-001, with the most significant increase being observed in patients remained in complete remission with stable MRD. In contrast, the level of cDC2 in patients who had relapse did not show significant change. In addition, the level of the cDC2 cells in patients who relapsed was overall significantly lower than those in patients who remained in complete remission with stable MRD and who had an MRD response.

3) Assessment of Dendritic Cells CD163+ cDC2 (HLA-DR+, CD14−, CD11b−, CD1c+, and CD163+) in Immune Response to Vaccination with DCP-001

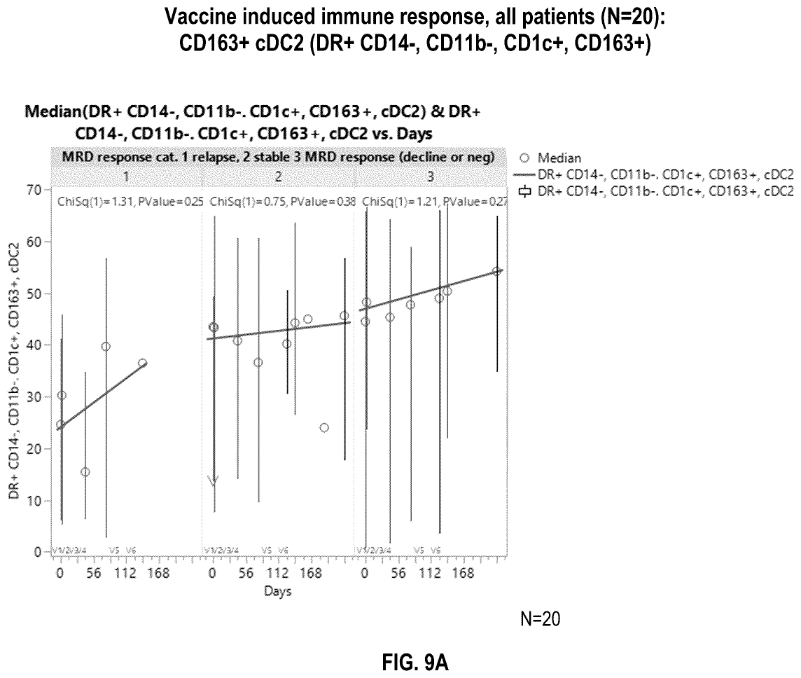

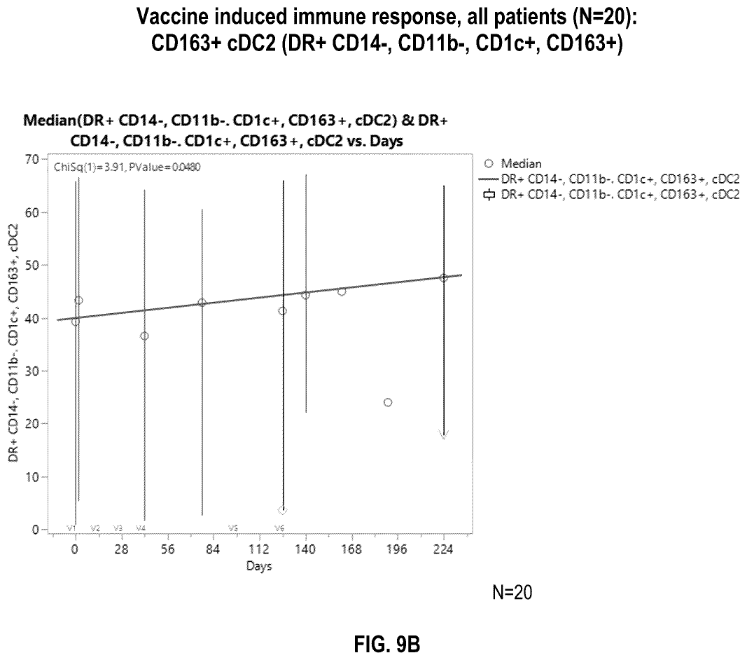

FIGS. 9A and 9B depict graphs illustrating the status of dendritic cells CD163+ cDC2 (DR+CD14− CD11b, CD1c+, and CD163+) in blood samples collected from the patients at time points before (V2), at the end of, and after the vaccination course with DCP-001. In FIG. 9, V2 corresponds to baseline (day 0), V7 corresponds to week 18, and V13 corresponds to week 32.

As shown in FIG. 9, the level of the CD163+ cDC2 cells in patients who remained in complete remission with stable MRD and who had an MRD response increased during and after the course of the vaccination with DCP-001. In contrast, the level of CD163+ cDC2 in patients who had relapse did not increase but even reduced at 18 weeks after the first dose of vaccination was administered. In addition, the level of the CD163+ cDC2 cells in patients who relapsed was overall significantly lower than those in patients who remained in complete remission with stable MRD and who had an MRD response.

The same assessment was performed to analyze HLA-DR+CD123+ pDC cells and similar results obtained. The level of the HLA-DR+CD123+ pDC cells in patients who remained in complete remission with stable MRD and who had an MRD response increased during and after the course of the vaccination with DCP-001. In contrast, the level of HLA-DR+CD123+ pDC in patients who had relapse decreased after at the end of the study and after, compared to baseline level. In addition, the level of the HLA-DR+CD123+ pDC cells in patients who had an MRD response was significantly higher than those in the patients who relapsed or who remained in complete remission with stable MRD.

4) Assessment of CD14+CD16− Non-Inflammatory Monocytes in Immune Response to Vaccination with DCP-001

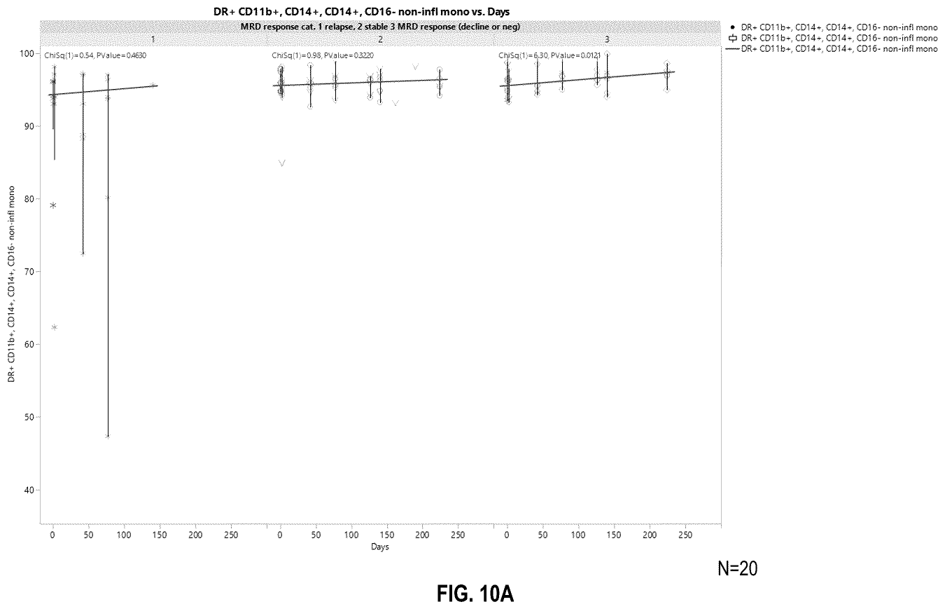

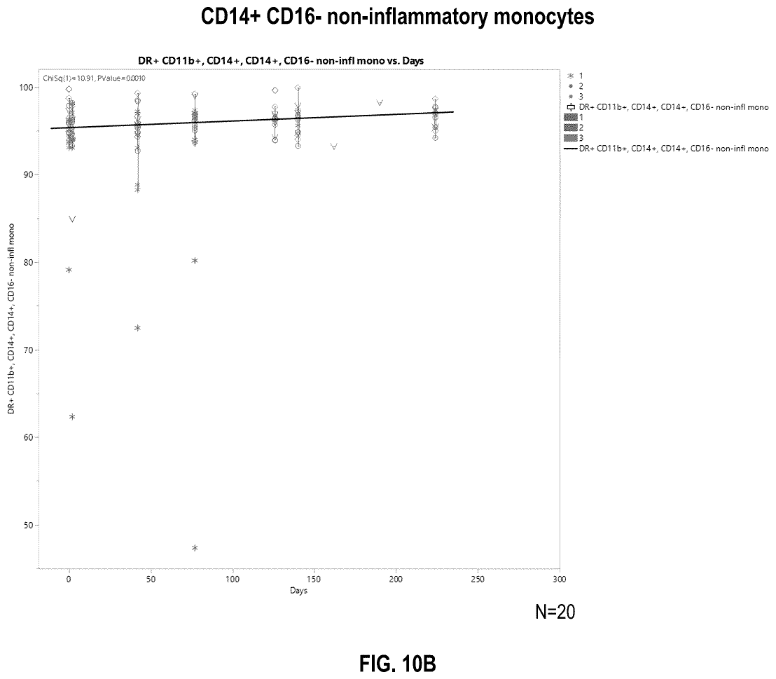

FIGS. 10A and 10B depict graphs illustrating the analysis of the change of level of CD14+CD16− non-inflammatory monocytes in blood samples collected from the patients before, during, and after the course of the vaccination with DCP-001 as described in Example 1. As can be seen in FIGS. 10A and 10B, the level of the CD14+CD16− non-inflammatory monocytes in patients who remained in complete remission with stable MRD and who had an MRD response increased during and after the course of the vaccination with DCP-001. In contrast, the level of CD14+CD16− non-inflammatory monocytes in patients who relapsed decreased during and after the vaccination.

5) Assessment of CD56++NK Cells in Immune Response to Vaccination with DCP-001

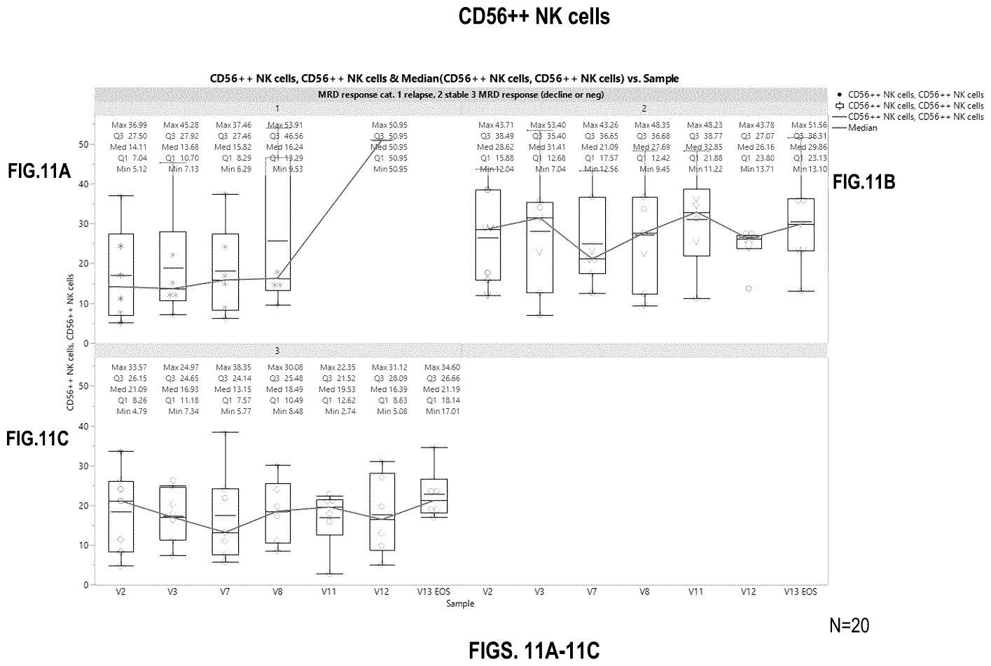

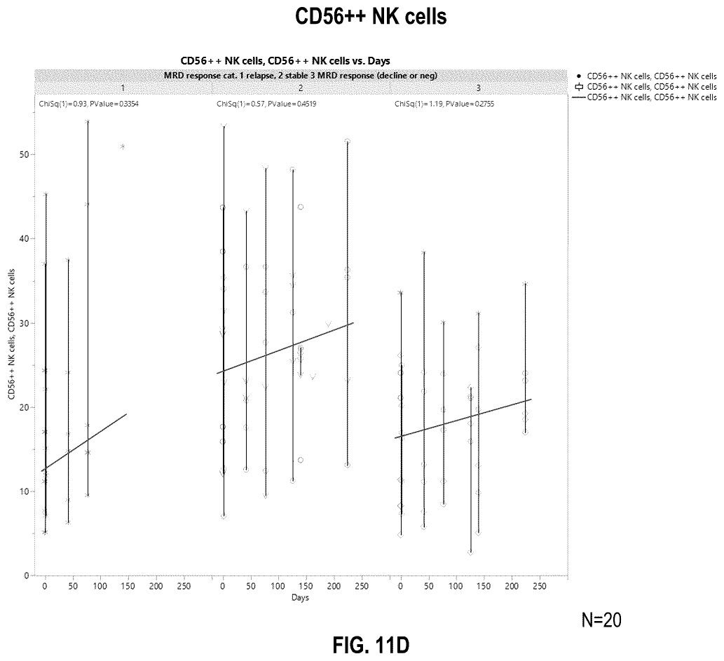

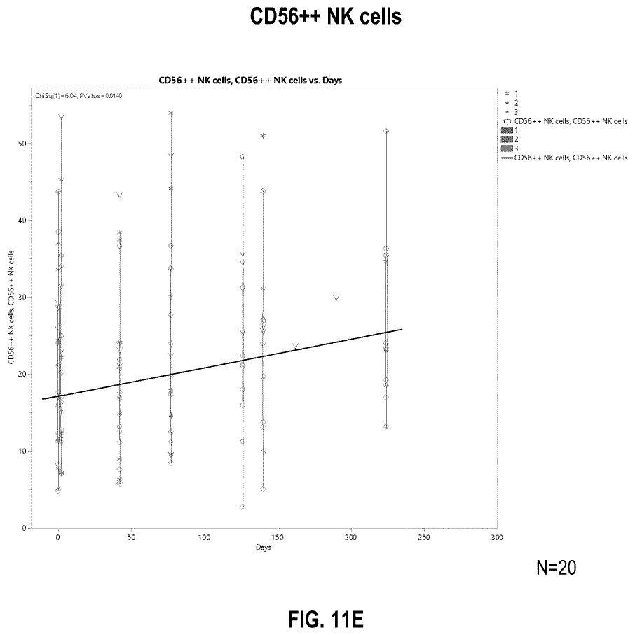

FIGS. 11A-11C depict graphs illustrating the status of CD56++NK in blood samples collected from the patients before, during, and after the course of the vaccination with DCP-001, as described in Example 1, where FIG. 11A illustrates the status of the CD56++NK cells in patients who relapsed, FIG. 11B illustrates the status of the CD56++NK cells in patients who remained in complete remission with stable MRD, and FIG. 11C illustrates the status of the CD56++NK cells in patients who had an MRD response. In FIGS. 11A-11C, V2 corresponds to baseline (day 0), V3 corresponds to day 3, V7 corresponds to day 42, V8 corresponds to day 77 (week 11), V11 corresponds to week 18, V12 corresponds to week 20, and V13 corresponds to week 32. FIGS. 11D and 11E illustrates the level of CD56++NK in the blood of the patients before, during, and after the course of the vaccination with DCP-001.

As shown in FIGS. 11A-11C, if only investing the media, no obvious changes in all patients before, during, and after the course of the vaccination are observed when the level of CD56++NK cells is analyzed based on sample. However, as shown in FIGS. 11D and 11E, the level of the CD56++NK cells, when being analyzed based on days, significantly increased in patients who remained in complete remission with stable MRD and who had an MRD response continuously, during and after the course of the vaccination with DCP-001, with the most significant increase being observed in patients remained in complete remission with stable MRD. In contrast, the level of CD56++NK in patients who had relapse did not show significant change. In addition, the level of the CD56++NK cells in patients who relapsed was overall significantly lower than those in patients who remained in complete remission with stable MRD and who had an MRD response.

6) Assessment of CD56+NK Cells in Immune Response to Vaccination with DCP-001

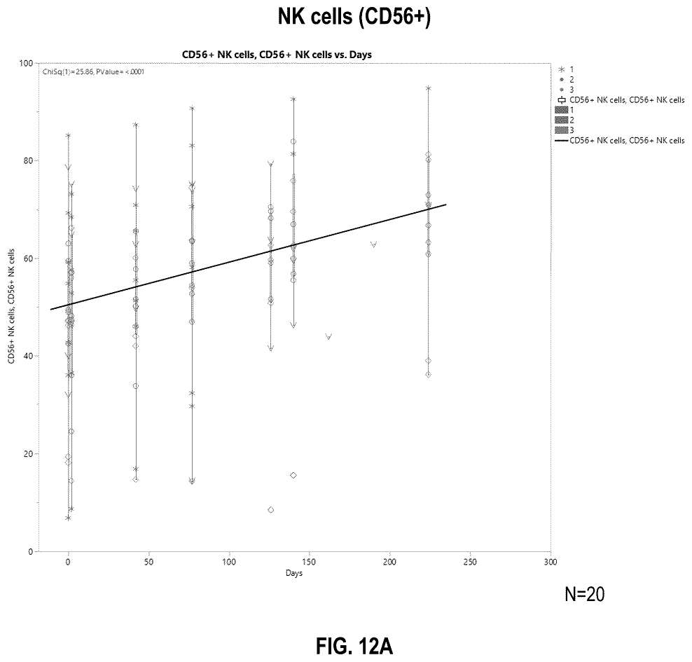

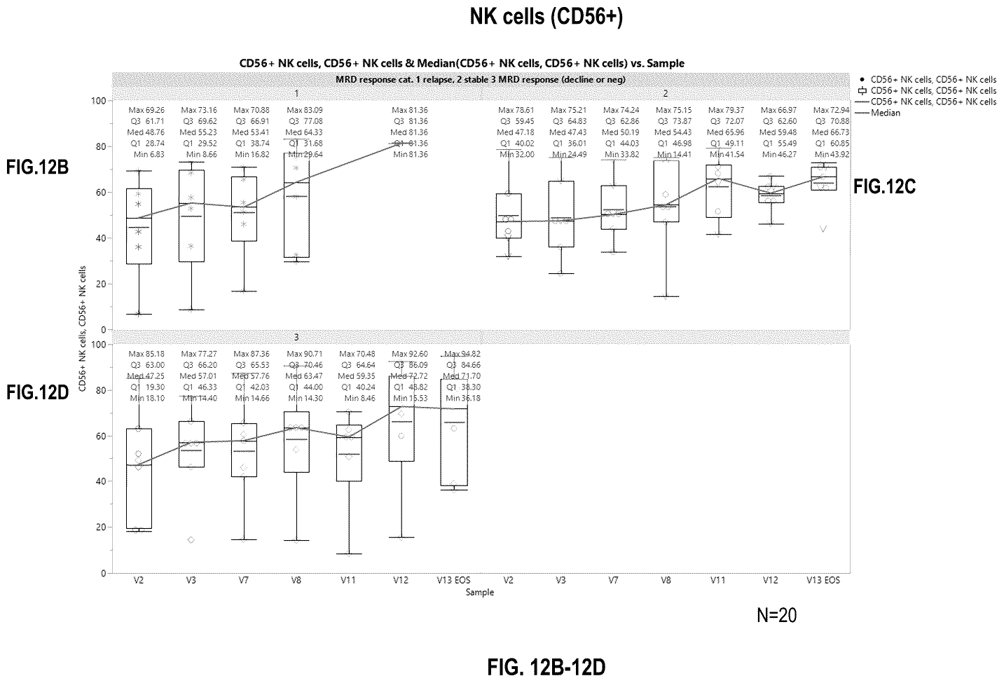

FIG. 12A illustrates the level of CD56+NK cells in the blood of the patients before, during, and after the course of the vaccination with DCP-001. FIGS. 12B-12D depict graphs illustrating the status of CD56+NK cells in blood samples collected from the patients before, during, and after the course of the vaccination with DCP-001, as described in Example 1, where FIG. 12B illustrates the status of the CD56+NK cells in patients who relapsed, FIG. 12C illustrates the status of the CD56+NK cells in patients who remained in complete remission with stable MRD, and FIG. 12D illustrates the status of the CD56+NK cells in patients who had an MRD response. In FIGS. 12B-12D, V2 corresponds to baseline (day 0), V3 corresponds to day 3, V7 corresponds to day 42, V8 corresponds to day 77 (week 11), V11 corresponds to week 18, V12 corresponds to week 20, and V13 corresponds to week 32.

As shown in FIGS. 12A-12D, the level of the CD56+NK cells in patients who remained in complete remission with stable MRD and who had an MRD response continuously increased during and after the course of the vaccination with DCP-001. In contrast, the level of CD56+NK in patients who had relapse did not show significant change. In addition, the level of the CD56+NK cells in patients who relapsed was overall significantly lower than those in patients who remained in complete remission with stable MRD and who had an MRD response.

7) Assessment of B Cells in Immune Response to Vaccination with DCP-001

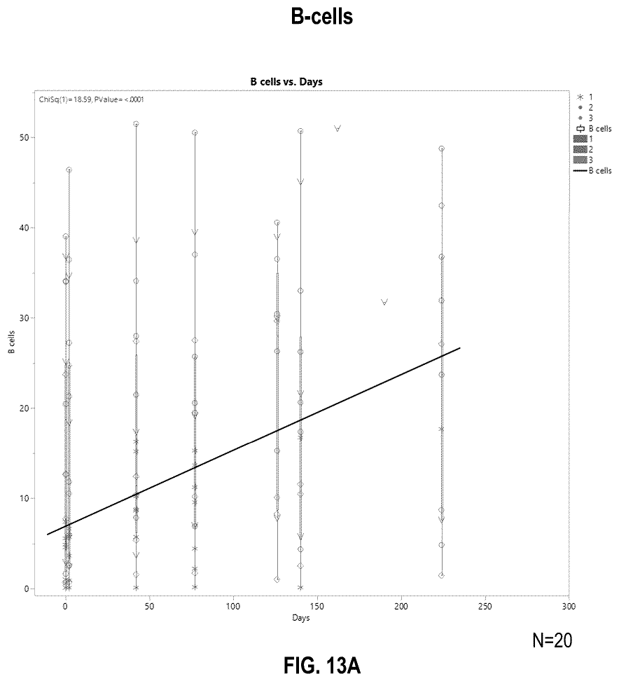

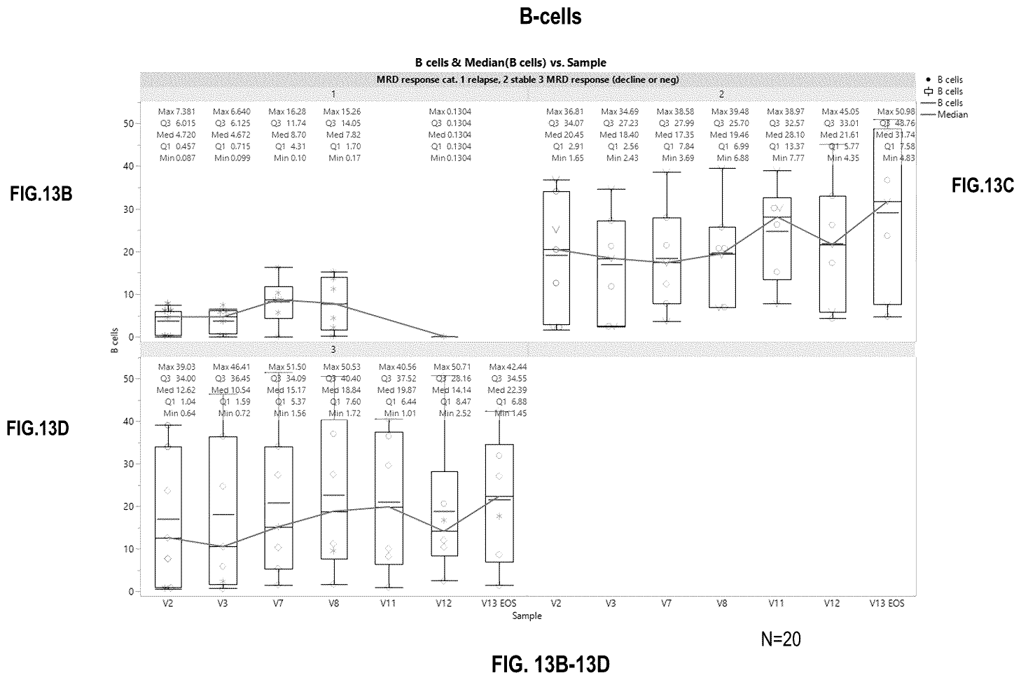

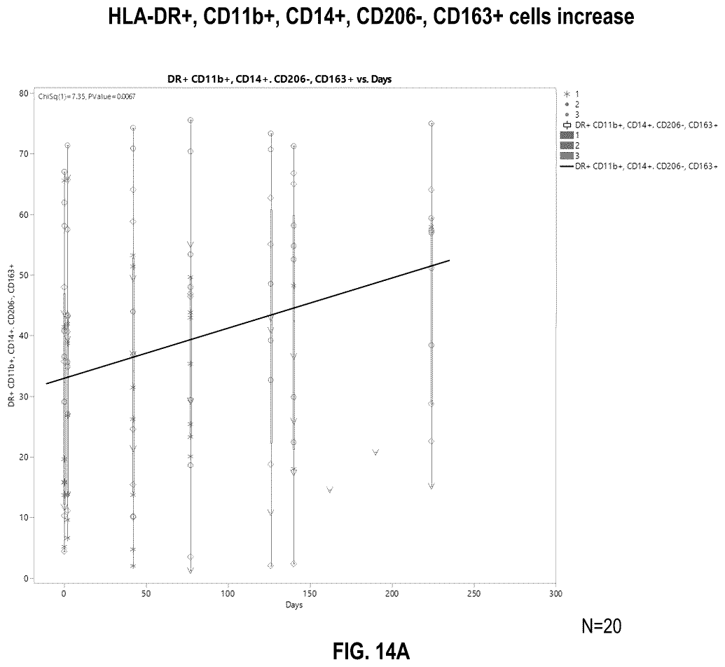

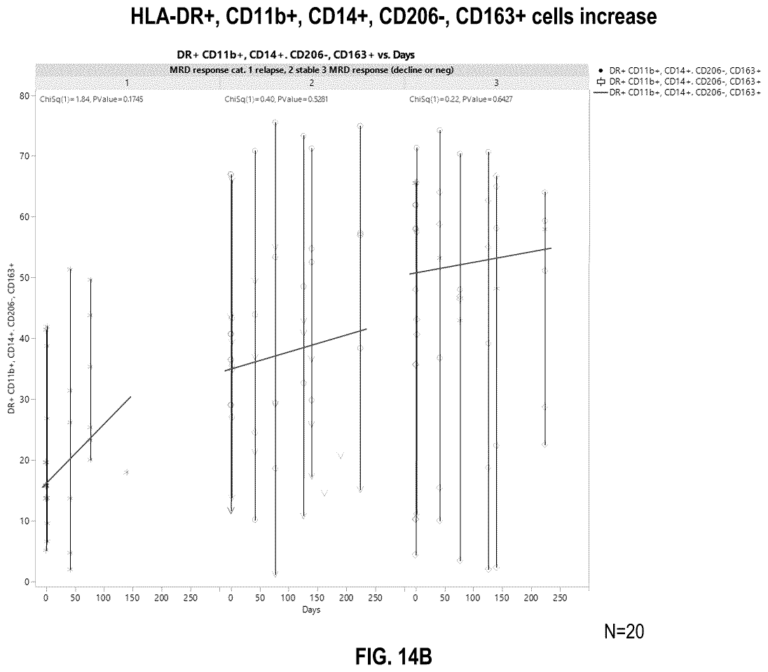

FIG. 13A illustrates analysis of the level of B cells in the blood of the patients before, during, and after the course of the vaccination with DCP-001. FIGS. 13B-13D depict graphs illustrating the status of B cells in blood samples collected from the patients before, during, and after the course of the vaccination with DCP-001, as described in Example 1, where FIG. 13B illustrates the status of the B cells in patients who relapsed, FIG. 13C illustrates the status of B cells in patients who remained in complete remission with stable MRD, and FIG. 13D illustrates the status of B cells in patients who had an MRD response. In FIGS. 13B-13D, V2 corresponds to baseline (day 0), V2 corresponds to baseline (day 0), V3 corresponds to day 3, V7 corresponds to day 42, V8 corresponds to day 77 (week 11), V11 corresponds to week 18, V12 corresponds to week 20, and V13 corresponds to week 32.

As shown in FIGS. 13A-13D, the level of B cells in patients who remained in complete remission with stable MRD and who had an MRD response continuously increased during and after the course of the vaccination with DCP-001. In contrast, the level of B in patients who relapsed did not show significant change. In addition, the level of the B cells in patients who relapsed was overall significantly lower than those in patients who remained in complete remission with stable MRD and who had an MRD response, even at baseline.

8) Assessment of HLA-DR+, CD11b+, CD14+, CD206−, CD163+ Cells in Immune Response to Vaccination with DCP-001

FIGS. 14A and 14B illustrate the level of HLA-DR+, CD11 b+, CD14+, CD206−, CD163+ cells in the blood of the patients before, during, and after the course of the vaccination with DCP-001. As can be seen in FIGS. 14A and 14B, the level of HLA-DR+, CD11b+, CD14+, CD206−, CD163+ cells in patients who remained in complete remission with stable MRD and who relapsed increased during and after the course of the vaccination with DCP-001, while the level was not significantly changed in patients who show an MRD response (MRD responders).

9) Assessment of CD16− CD56+NK Cells in Immune Response to Vaccination with DCP-001

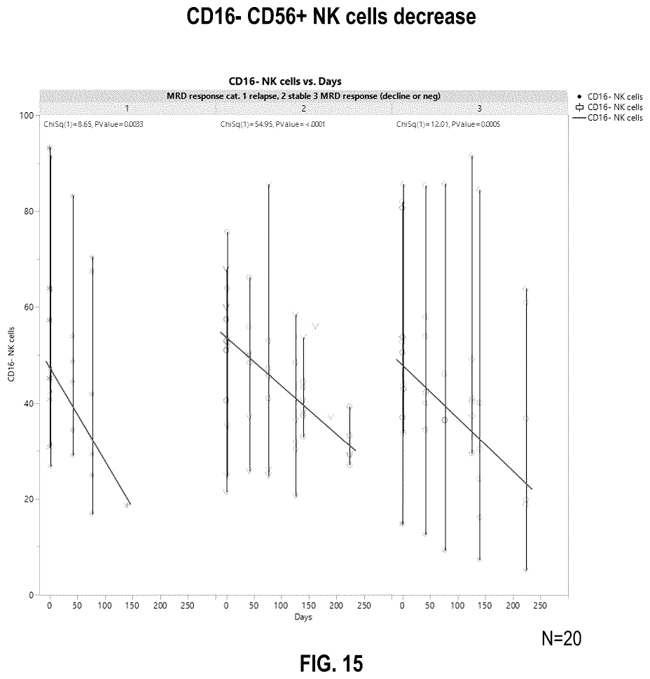

FIG. 15 illustrates the level of CD16-CD56+NK cells in the blood of the patients before, during, and after the course of the vaccination with DCP-001. As can be seen in FIG. 15, the level of CD16-CD56+NK cells in all patients decreased during and after the vaccination with DCP-001.

10) Assessment of CD4 LAG3+ Cells in Immune Response to Vaccination with DCP-001

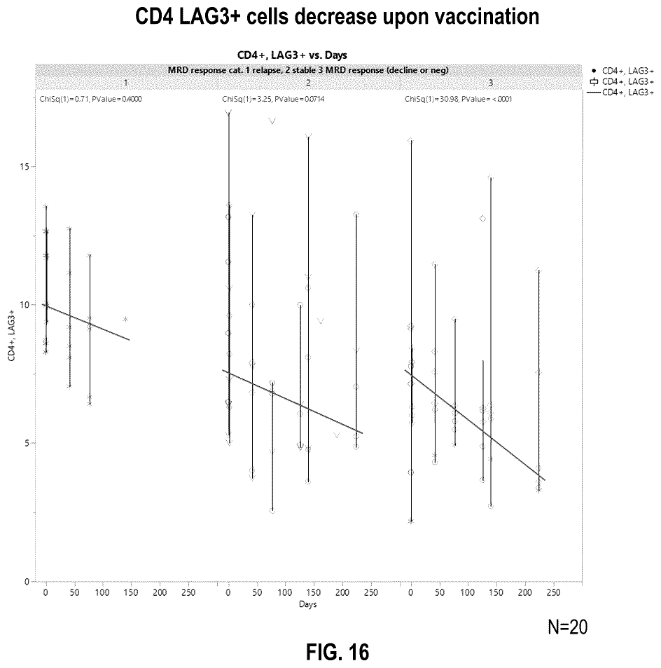

FIG. 16 illustrates the level of CD4+ LAG3+ cells in the blood of the patients before, during, and after the course of the vaccination with DCP-001. As can be seen in FIG. 16, the level of CD4+ LAG3+ cells in all patients decreased during and after the vaccination with DCP-001.

2.3 Evaluation of Dendritic Cell Subsets Upon Vaccination with DCP-001

Background. Active immunotherapy relies on the patient's immune system for induction of an effective immune response. Not only the frequency, number and exhaustion or activation status of CD8 T cells is important for an efficacious tumor cell lysis, but the quality, number and distribution of different antigen presenting cells is of equal importance. In this study circulating dendritic cells and monocytes have been investigated before and during vaccination to investigate the effect of vididencel treatment on the composition of antigen presenting cells in relation to clinical responses.

Methods. The patients and vaccination strategies/administration schedule are the same as described above in Example 1. Peripheral blood mononuclear cells were taken at baseline, week 6, 11, 14, 18, 20 and 32, Ficoll isolated and cryopreserved in liquid nitrogen. Analysis of immune cell composition was performed by spectral flow cytometry, using a single 40-marker panel. Immune subset analysis of dendritic (live, single, CD45+, Lin-(CD3, 56, 19), CD16−, CD15−) cells was performed and correlated to clinical response, relapse free and overall survival, by high dimensional analysis using Flow SOM.

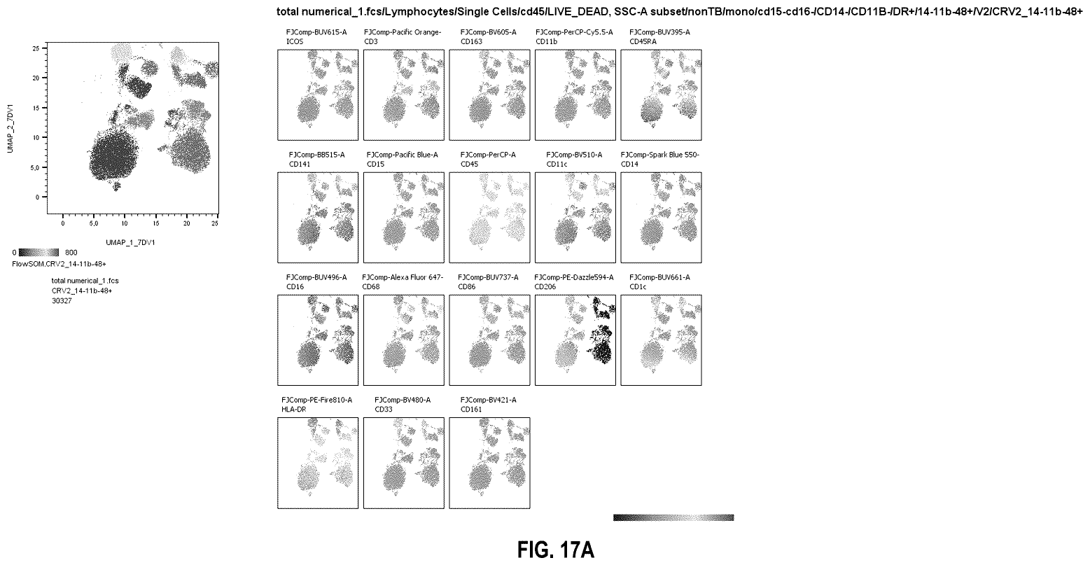

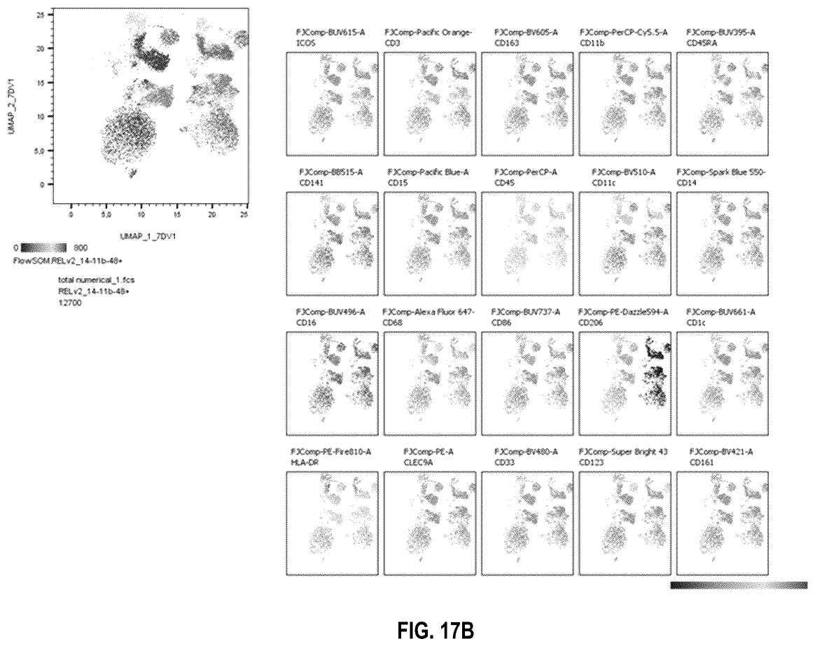

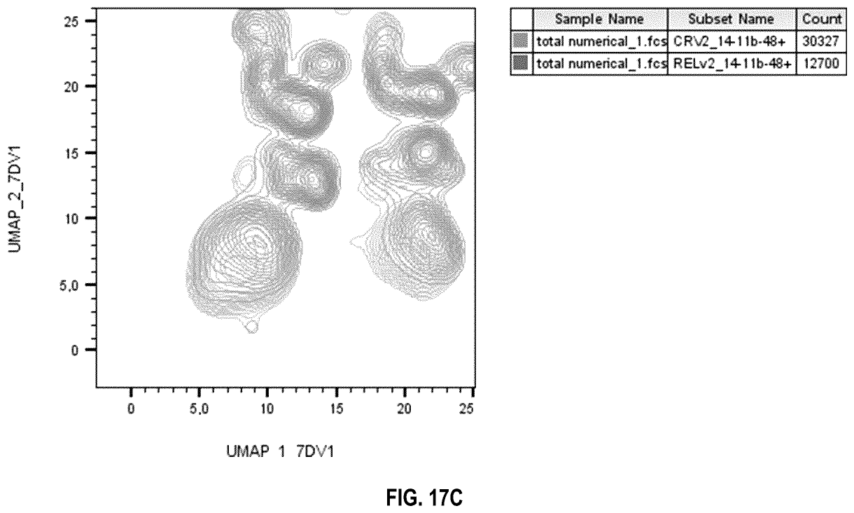

Results. The results are illustrated in FIG. 17A-FIG. 17C, which depict a Uniform Manifold Approximation and Projection (UMAP) plot of CD45, CD3−, CD19−, CD56−, CD14−, CD15−, CD16−, CD11b−, HLA-DR+ lymphocytes. Dendritic cell subsets increased upon vaccination, with levels before and during vaccination being the highest in patients remaining in CR. Gating of the dendritic cell subsets by spectral flow cytometry analyses could be hampered by presence of leukemic blasts in the peripheral blood, expressing for example CD123, a marker required for gating of pDCs. Using gating on the lineage negative, CD15−, CD16− and HLA-DR+ cells, a deep immunophenotyping was performed on dendritic cells in peripheral blood. Baseline analysis showed a significantly higher frequency of CD45RA+, HLA-DR+ cells in patients who remain in CR. Using tSNE and UMAP plots generated to analyze changes in the Lin−, CD14−, CD15−, CD16− and CD11b−, HLA-DR+ population.

Changes and increases in several subsets were observed, most notably in subsets with cDC1, cDC2 and pDC characteristics, like expression of CD141, CLEC9A, CD1c and CD123. Higher frequencies at baseline of dendritic cells (cDC1 and cDC2) correlated positively with both relapse-free and overall survival. In patients remaining in CR, vaccination further increased or maintained dendritic cell subset frequencies, of which classical cDC1 and cDC2 correlated with longer overall survival (p<0.05).

Without intending to be bound by scientific theory, patients who remained in CR after vididencel treatment had the highest baseline levels of HLA-DR+CD45RA+ cells, which could be myeloid cells able to differentiate into dendritic cell subsets, as in general CD45RA+ is lost during maturation from precursor to matured dendritic cells. Vaccination might improve and induce maturation of dendritic cell subsets, such as cDC1, cDC2 and pDC, which enhanced antigen capturing, processing and presentation to tumor-reactive T cells, ultimately leading to improved survival.

### CONCLUSION/DISCUSSION

The above flow cytometry analysis for baseline immune cell composition in the peripheral blood samples showed that patients who showed a decrease in MRD or conversion to MRD negativity had the highest level of tumor-specific and sustained VIR accompanied by the highest level of baseline DCs and lowest level of CD8 CM in the peripheral blood. Patients who remained in complete remission after vaccination showed a higher frequency of cDC2, CD4+/CD161+ T cells, and B cells, and lower frequency of NK cells and CD8+ central memory T cells at baseline, compared to patients with clinical relapse. Patients who eventually relapsed had low levels of B-cells and lower levels of cDC1/cDC2 at baseline, and higher levels of CD8 central memory and NK-cells at baseline.

In patients with stable MRD, the total number of circulating DC increased after vaccination compared to baseline, while these circulating DC remained at comparable level in the responders. CD8 memory profiles indicated a difference in baseline levels of CD8 RO+(memory T cells), with far higher number of especially CD8 CM in patients who relapsed. In addition, the immune cell profile during vaccination was characterized by an increased frequency of cDC1, cDC2 and NK cells and decreased frequency of LAG3+CD4+ T cells in patients who remained in complete remission, compared to patients with clinical relapse. Patients who relapsed also showed increase in immune cell levels, but the levels remained low relative to patients who stayed in complete remission.

These findings indicate that the immune status pre-vaccination affect the clinical response to DCP-001 vaccination and that those patients who remains in complete clinical remission after vaccination show increased frequency of cDC1, cDC2 and NK cells and lower frequency of CD4+ T cells expressing the exhaustion marker LAG-3.

Based on these data, not just cDC1, cDC2 or pDC is important to exert an effect in a vaccination-based therapy for AML maintenance, but rather the full composition of these DCs. This underscores the importance of cross-priming CD8 by either of these DCs. The vaccination induced a change in the DC compartment for patients which had a stable MRD response at week 32, with increasing levels DCs after vaccination. Although the number in the peripheral blood is low, this might reflect the need of a certain level of DCs, for an even better immune response. The number of DCs as such might also require sufficient CD8 effector cells to either respond or to be primed after vaccination as reflected by low CD8 memory and high number of VIR in MRD responders.

The use of an allogenic dendritic cell-based vaccine induces a local and systemic immune response, in AML patients in CR but with MRD. Higher levels of circulation DCs in combination with CD8 effector cells, instead of high CD8 CM might be a prerequisite for successful vaccination to prevent relapse or MRD conversion. Improvement in DC levels were observed in patients with stable MRD, which might render a suggest that for more booster vaccinations using this vaccine.

The data shows that the composition and number of certain immune cells in the peripheral blood can be used as biomarkers to predict a successful MRD response to relapse vaccination strategies in liquid cancer patients. Levels and phenotypes of these circulating immune cells can be measured before vaccination to screen patients before starting relapse vaccination strategies or help with design vaccination strategies or regimens. For example, such circulating immune cells may be chosen from dendritic cells such as cDC1, cDC2, and pDC, CD8 T cells such as CD8+CD45RA+ cells, CD8+CD45RA− CCR7+, CM T cells, and CD8 RO+ T cells, B cells, NK cells, CD4 CD161+ T cells, CD14+CD16− non-inflammatory monocytes, or any combination thereof, where the dendritic cells may be conventional dendritic cells DC1 (cDC1) such as CD141+/CLEC9A+ cDC1, conventional DC2 (cDC2) such as CD163+ cDC2, and/or plasmacytoid dendritic cells (pDC), or a combination thereof, the CD8 T cells may be CD8+CD45RA+ cells, CD8+CD45RA− CCR7+CM T cells, and/or CD8 RO+ T cells, and the NK cells may be CD56++NK cells and/or CD56+NK cells.

As such, in some situations, a baseline immune cell composition in blood can be used as a parameter to select a patient to treat with DCP-001 and/or predict vaccination results, where a low level of certain immune cells, such as B-cells and lower levels of cDC1/cDC2 at baseline, or a higher level of certain immune cells such as CD8 central memory and NK-cells at baseline can indicate that a patient would eventually relapse even after treatment with DCP-001. In some other situations, DCP-001-induced changes in different immune cell subsets during vaccination can be used a parameter to help with selecting patients or designing vaccination schedules to treat MRD. For instance, an increase of certain immune cells, for example, B cells, cDC1, cDC2, NK-cells, during vaccination may be used as a parameter to select patient for vaccination and/or continuous vaccination.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A method of treating liquid cancer in a subject in need thereof, comprising administering to a subject having measurable residual disease (MRD) and an altered level of a biomarker in peripheral blood of the subject relative to a reference level of the biomarker, a composition comprising an allogeneic leukemia-derived cell,
wherein the biomarker is chosen from dendritic cells, CD8+ T cells, B cells, NK cells, CD4 CD161+ T cells, CD14+CD16− non-inflammatory monocytes, or a combination thereof.

2. The method of claim 1, wherein the dendritic cells are selected from the group consisting of conventional dendritic cells DC1 (cDC1), conventional DC2 (cDC2), plasmacytoid dendritic cells (pDC), or a combination thereof, wherein the level of the cDC1, cDC2, and/or pDC cells is elevated relative to a reference level of the biomarker.

3. The method of claim 1, wherein the dendritic cells comprise CD141+/CLEC9A+ cDC1 cells, wherein the altered level comprises an increased level of the CD141+/CLEC9A+ cDC1 cells relative to the reference level of the biomarker.

4. The method of claim 1, wherein the dendritic cells comprise cDC2 and/or CD163+ cDC2 dendritic cells, wherein the altered level comprises an increased level of the cDC2 and/or CD163⁺ cDC2 dendritic cells relative to the reference level of the biomarker.

5. The method of claim 1, wherein the dendritic cells comprise HLA-DR+/CD123+ pDC cells, wherein the altered level comprises an elevated level of the HLA-DR+/CD123+ pDC cells relative to the reference level of the biomarker.

6. The method of claim 1, wherein the NK cells comprise CD56++NK cells and/or CD56+NK cells, and wherein the altered level comprises a decreased level of CD56+NK, and/or an elevated level of CD56++NK cells, relative to the reference level of the biomarker.

7. The method of claim 1, wherein the CD8+ T cells comprise CD8+/CD45RA+ T cells, CD8+/CD45RA−/CCR7+CM T cells, and/or CD8/RO+ T cells, wherein the altered level comprises a decreased level of CD8+/CD45RA−/CCR7+CM T cells and/or CD8 RO+ T cells, and/or an elevated level of CD8+/CD45RA+ T cells relative to the reference level.

8. The method of claim 1, wherein the altered level of the biomarker is a baseline level of the biomarker in the subject, and the reference level is a baseline level of the biomarker in another subject who displays relapse after treating with the composition comprising the allogeneic leukemia-derived cell.

9. The method of claim 1,
wherein the liquid cancer is selected from the group consisting of leukemia, lymphoma, myelodysplastic syndrome (MDS), myeloma, and a combination thereof.

10. The method of claim 1, wherein the liquid cancer is acute myeloid leukemia (AML).

11. The method of claim 1,
(a) wherein the allogeneic leukemia-derived cell comprises WT-1, MUC-1, PRAME, RHAMM, p53, and Survivin;
(b) wherein the allogeneic leukemia-derived cell comprises a dendritic cell phenotype;
(c) wherein the allogeneic leukemia-derived cell comprises a mature dendritic cell phenotype;
(d) wherein the allogeneic leukemia-derived cell comprises a genetic aberration between chromosome 11p15.5 to 11p12, optionally wherein the genetic aberration encompasses about 16 Mb of genomic regions;
(e) wherein the allogeneic leukemia-derived cell expresses a cell surface marker selected from the group consisting of DC-SIGN, Langerin, CD80, CD86, CD70, CD40, and any combination thereof;
(f) wherein the allogeneic leukemia-derived cell is CD34-positive, CD1a-positive, CD83-positive, CD80-positive, CD86-positive, and CD40-positive; and/or
(g) wherein the allogeneic leukemia-derived cell is CD14-negative.

12-15. (canceled)

16. The method of claim 1, wherein the allogeneic leukemia-derived cell is CD34-positive, CD1a-positive, and CD83-positive.

17-19. (canceled)

20. The method of claim 1, wherein the allogeneic leukemia-derived cell is derived from the DCOne cell line.

21. The method of claim 1, wherein:
(a) the subject receives one or more biweekly doses of about 25e6 or about 50e6 allogeneic leukemia-derived dendritic cells;
(b) the composition is administered to the subject by injection that is optionally intradermal;
(b) the subject achieves MRD conversion or disappearance;
(c) the subject had previously been treated; and/or
(d) the subject is in complete remission (CR).

22. The method of claim 21, wherein the subject receives one or more booster doses, each dose comprising about 10e6 allogeneic leukemia-derived dendritic cells, optionally each booster dose being administered to the subject about 28 days after a previous dose.

23-27. (canceled)

28. A method of treating cancer in a subject in in need thereof, comprising:
administering to a subject having measurable residual disease (MHRD) one or more initial doses of an allogeneic leukemia-derived dendritic cell vaccine; and
administering to the subject one or more booster doses of the allogeneic leukemia-derived dendritic cell vaccine, if an elevated level of a biomarker is achieved in the subject subsequent to the one or more initial doses of the allogeneic leukemia-derived dendritic cell vaccine relative to the biomarker level in the subject prior to the one or more initial doses, wherein the biomarker comprises is chosen from dendritic cells comprising conventional dendritic cells DC1 (cDC1), conventional DC2 (cDC2), plasmacytoid dendritic cells (pDC), or a combination thereof, CD8+CD45RA+ cells, B cells, NK cells, CD4 CD161+ T cells, CD14+CD16− non-inflammatory monocytes, or a combination thereof.

29. The method of claim 28, wherein the dendritic cells comprise HLA-DR+/CD123+ pDC cells.

30. The method of claim 28, wherein the dendritic cells comprise CD141+/CLEC9A⁺ cDC1 dendritic cells.

31. The method of claim 28, wherein the dendritic cells comprise CD163⁺ cDC2 cells.

32. The method of claim 28, wherein the NK cells comprise CD56++NK cells and/or CD56+NK cells.

33. The method of claim 28, wherein
(a) the cancer is a liquid cancer, optionally wherein the liquid cancer is selected from the group consisting of leukemia, myelodysplastic syndrome (MDS), lymphoma, myeloma, and combination thereof;
(b) the liquid cancer is selected from the group consisting of leukemia, myelodysplastic syndrome (MDS), lymphoma, myeloma, and combination thereof, optionally wherein the liquid cancer is acute myeloid leukemia (AML);
(c the subject receives the one or more initial doses of vaccine biweekly, and wherein the one or more initial doses of vaccine each comprises about 25e6 or about 50e6 allogeneic leukemia-derived dendritic cells;
(d) the one or more booster doses of vaccine each comprises about 10e6 allogeneic leukemia-derived dendritic cells, optionally each booster dose is administered to the subject about 28 days after a previous dose;
(e) wherein the subject achieves MRD conversion or disappearance;
(f) wherein the composition is administered to the subject by injection.
(g) wherein the subject had previously been treated; and/or
(h) wherein the subject is in complete remission (CR).

34. (canceled)

35. The method of claim 28, wherein the cancer is acute myeloid leukemia (AML).

36. The method of claim 28, wherein:
(a) the allogeneic leukemia-derived cell comprises WT-1, MUC-1, PRAME, p53, RHAMM, and Survivin;
(b) the allogeneic leukemia-derived cell comprises a dendritic cell phenotype;
(c) the allogeneic leukemia-derived cell comprises a mature dendritic cell phenotype;
(d) the allogeneic leukemia-derived cell comprises a genetic aberration between chromosome 11p15.5 to 11p12, optionally wherein the genetic aberration encompasses about 16 Mb of genomic regions;
(e) the allogeneic leukemia-derived cell expresses a cell surface marker selected from the group consisting of DC-SIGN, Langerin, CD80, CD86, CD70, CD40, and any combination thereof
(f) the allogeneic leukemia-derived cell is CD34-positive, CD1a-positive, CD83-positive, CD80-positive, CD86-positive, and CD40-positive; and/or
(g) the allogeneic leukemia-derived cell is CD14-negative.

37-40. (canceled)

41. The method of claim 28, wherein the allogeneic leukemia-derived cell is CD34-positive, CD1a-positive, and CD83-positive.

42-44. (canceled)

45. The method of claim 28, wherein the allogeneic leukemia-derived cell is derived from the DCOne cell line.

46-52. (canceled)

53. A method of identifying a subject with cancer in remission having measurable residual disease (MRD) who is likely to have a stable MRD or MRD conversion in response to a treatment with an immunogenetic composition, predicting a risk of developing cancer relapse or recurrence, and/or screening a candidate to receive the treatment with the immunogenetic composition, the method comprising:
assessing a baseline level of a biomarker in a peripheral blood of the subject before the subject is treated with the immunogenetic composition in comparison with a predetermined reference level of the biomarker; and
identifying the subject as being likely to have a stable MRD or MRD conversion in response to the treatment with the immunogenetic composition if the baseline level of the biomarker in the peripheral blood of the subject is altered relative to the predetermined reference level of the biomarker;
wherein the immunogenetic composition comprises allogeneic leukemia-derived dendritic cells; and
wherein the biomarker is chosen from dendritic cells, CD8+ T cells, B cells, NK cells, CD4 CD161+ T cells, CD14+CD16− non-inflammatory monocytes, or a combination thereof.

54-57. (canceled)

58. A method of identifying a subject with cancer in remission having measurable residual disease (MRD) who is likely to have a stable MRD or MRD conversion in response to a treatment with an immunogenetic composition, predicting a risk of developing cancer relapse or recurrence, and/or identifying a need of a continuation treatment with the immunogenetic composition to prevent or delay relapse or recurrence of cancer or reduce the risk of developing cancer relapse, the method comprising:
assessing a baseline level of a biomarker in peripheral blood of the subject before treating the subject with the immunogenetic composition;
assessing one or more post-treatment levels of the biomarker in peripheral blood of the subject after the subject is treated with the immunogenetic composition according to a first dosage regimen; and
identifying the subject as in need of continuous treatment with the immunogenetic composition to prevent or delay relapse or recurrence of cancer if the one or more post-treatment levels of the biomarker are greater than the baseline level of the biomarker;
wherein the immunogenetic composition comprises allogeneic leukemia-derived dendritic cells; and
wherein the biomarker is chosen from dendritic cells, CD8+ T cells, B cells, NK cells, CD4 CD161+ T cells, CD14+CD16− non-inflammatory monocytes, or a combination thereof.

59-61. (canceled)

62. A method of identifying a subject with cancer in remission having measurable residual disease (MRD) as being likely to have a stable MRD or MRD conversion in response to a treatment with an immunogenetic composition and treating the identified subject to prevent or delay relapse or recurrence of cancer, and/or reduce a risk of relapse or recurrence of cancer, the method comprising:
assessing a baseline level of a biomarker in a peripheral blood of a subject before the subject is treated with the immunogenetic composition in comparison with a predetermined reference level of the biomarker;
identifying the subject as being likely to be responsive to the treatment with the immunogenetic composition if the level of baseline level of the biomarker in the peripheral blood of the subject is altered compared to the predetermined reference level of the biomarker; and
administering to the identified subject with at least one dose of the immunogenetic composition comprising an effective amount of allogeneic leukemia-derived dendritic cells;
wherein the biomarker is chosen from dendritic cells, CD8+ T cells, B cells, NK cells, CD4 CD161+ T cells, CD14+CD16− non-inflammatory monocytes, or a combination thereof.

63. (canceled)

64. A method of identifying a subject with cancer in remission having measurable residual disease (MRD) who has been treated with an immunogenetic composition according to a first dosage regimen and is likely in need of continuous or booster treatment with the immunogenetic composition, and treating the subject to prevent or delay relapse or recurrence of cancer, and/or reduce a risk of relapse or recurrence of cancer, the method comprising:
assessing a baseline level of a biomarker in peripheral blood of the subject before the subject is treated with the immunogenetic composition;
assessing one or more post-treatment levels of the biomarker in peripheral blood of the subject after the subject is treated with the immunogenetic composition according to a first dosage regimen;
identifying the subject as a candidate to receive continuous treatment with the immunogenetic composition to prevent or delay relapse or recurrence of cancer if the one or more post-treatment levels of the biomarker are altered relative to the baseline level of the biomarker; and
administering to the identified subject with the immunogenetic composition according to a second booster regimen;
wherein the immunogenetic composition comprises an allogeneic leukemia-derived cell; and
wherein the biomarker is chosen from dendritic cells, CD8+ T cells, B cells, NK cells, CD4 CD161+ T cells, CD14+CD16− non-inflammatory monocytes, or a combination thereof.

65-74. (canceled)

75. The method of claim 53, wherein the biomarker is chosen from dendritic cells comprising conventional dendritic cells CD1 (cDC1), conventional DC2 (cDC2), plasmacytoid dendritic cells (pDC), CD8+ T cells comprising CD8+CD45RA+ cells, CD8+/CD45RA−/CCR7+CM T cells, and/or CD8/RO+ T cells, B cells, NK cells, CD4 CD161+ T cells.

76. The method of claim 75, wherein the dendritic cells comprise HLA-DR+/CD123+ pDC cells.

77. The method of claim 75, wherein the dendritic cells comprise CD141⁺/CLEC9A⁺ cDC1 dendritic cells.

78. The method of claim 75, wherein the dendritic cells comprise CD163⁺ cDC2 dendritic cells.

79. The method of claim 75, wherein the NK cells comprise CD56++NK cells and/or CD56+NK cells.

80-86. (canceled)

87. The method of claim 53, wherein the allogeneic leukemia-derived cells are CD34-positive, CD1a-positive, and CD83-positive cells and comprise a non-tumor antigen or a nucleic acid encoding the non-tumor antigen.

88. The method of claim 53, wherein the allogeneic leukemia-derived cells are derived from DCOne cell line.

89-90. (canceled)

91. The method of claim 53, wherein the cancer is acute myeloid leukemia (AML).

92. The method of claim 53, wherein the subject is ineligible for accepting hematopoietic stem cell transplantation or wherein the subject has been initially treated with a chemotherapy and/or debulking surgery, and the remission is induced by the chemotherapy and/or the debulking surgery.

93-95. (canceled)

96. The method of claim 1, wherein the subject has an elevated baseline level of HLA-DR+CD45RA+ dendritic cells relative to a reference level of the biomarker.
